(19) United States
(12) Patent Application Publication
(10) Pub. No.: US 2018/0318336 A1
(43) Pub. Date: Nov. 8, 2018

(54) PENTOSAN POLYSULFATE SODIUM FOR THE TREATMENT OF SICKLE CELL DISEASE

(71) Applicant: Vanguard Therapeutics, Inc., Half Moon Bay, CA (US)

(72) Inventor: Stephen H. Embury, Half Moon Bay, CA (US)

(21) Appl. No.: 15/576,616

(22) PCT Filed: May 27, 2016
(86) PCT No.: PCT/US16/34691

§ 371 Date: Nov. 22, 2017

Related U.S. Application Data
(60) Provisional application No. 62/166,863, filed on May 27, 2015.
(60) Provisional application No. 62/245,766, filed on Oct. 23, 2015.

(51) Int. Cl. A61K 31/737; C08B 37/00; A61P 7/00

(52) U.S. Cl. CPC A61K 31/737; G01N 33/94; A61P 7/00; C08B 37/0057

(57) ABSTRACT

This invention is directed to, inter alia, compositions comprised of pentosan polysulfate sodium (PPS) components and methods for making such compositions and using the same for the treatment of sickle cell disease (SCD). The disclosed compositions possess superior bioavailability as well as P-selectin blocking activity for the treatment of sickle-cell disease (SCD). Methods for using the same are additionally provided herein. Also provided herein is a method for detecting or quantifying PPS or a PPS fraction in solutions or in a biological sample obtained from an animal or an individual.

### CROSS-REFERENCE TO RELATED APPLICATIONS

This application is a national stage application filed under 35 U.S.C. § 371, of International Patent Application No. PCT/US2016/034691, filed May 27, 2016, which claims the benefit of and priority under 35 U.S.C. § 119(e) to U.S. Provisional Patent Application No. 62/166,863, filed May 27, 2015 and U.S. Provisional Patent Application No. 62/245,766, filed Oct. 23, 2015, the disclosures of each of which are hereby expressly incorporated by reference herein in their entireties.

### GOVERNMENT INTEREST

The invention was made with government support under R43 HL123059 awarded by The National Heart Lung and Blood Institute of the National Institutes of Health. The government has certain rights in the present invention.

### FIELD OF INVENTION

This invention is directed to, inter alia, compositions comprised of pentosan polysulfate sodium (PPS) components and methods for making such compositions and using the same for the treatment of sickle cell disease (SCD).

### BACKGROUND

Sickle-cell disease (SCD), also known as sickle-cell anemia (SCA, which more precisely is used to identify the most common genotypes that causes SCD, homozygosity for HbSS) and drepanocytosis (the Greek name for the disease), is an autosomal recessive genetic blood disorder caused by a point mutation in the β-globin chain of hemoglobin. SCD is characterized by red blood cells that adopt an abnormal, rigid, sickle shape, referred to as “sickling” under low-oxygen conditions. The abnormal cells with the potential to sickle are referred to as sickle red blood cells (SRBC). Repeated episodes of sickling can damage the blood cell's membrane and decrease its deformability. Some of the sickled cells can fail to return to normal shape when normal oxygen tension is restored. These irreversibly sickled SRBC and reversibly sickled SRBC are rigid and unable to deform as they pass through narrow capillaries, leading to vessel occlusion and ischemia. The actual anemia of the illness is caused mainly by hemolysis, the premature destruction of the SRBC.

P-selectin is a 140-kDa protein that is commonly expressed by platelets and endothelial cells (See, for example, GenBank Accession No. P16109 (Homo sapiens) or GenBank Accession No. AAA40008 (Mus musculus). P-selectin plays an essential role in the initial recruitment of leukocytes (white blood cells) to a site of injury during inflammation. The importance of P-selectin to the pathophysiology of SCD is understood through its effect on the adhesion of SRBC to the vascular endothelium and on the consequent impairment of blood flow. P-selectin is central to the abnormal blood flow in SCD, and abnormal blood flow is paramount to the morbidity and mortality of the disorder.

Pentosan polysulfate sodium (PPS) is an orally absorbable semisynthetic sulfated polysaccharide that is composed of chains of β-D-xylose with sulfated groups on C2 and C3 with 4-0 methyl D glucuronic acid (with sulfated groups on C2 and C3) associated in a lateral position in the chain on average every eight to ten xyloses (Maffrand, et al., (1991) Semin Thromb Hemost 17, Suppl 2, 186-198). It has structural and functional similarities to but considerably less anticoagulant activity than heparin, as well as potent blocking activity against SRBC adhesion to P-selectin (Kutlar, et al., (2012) Am J Hematol 87, 536-539 plus Online Supplemental Material). However, commercially available PPS is not an ideal therapy for SCD because of its marginal oral bioavailability, heterogeneity, and limited duration of action. What is needed, therefore, is a PPS-containing composition that is more homogeneous and having the properties of both potent P-selectin blocking activity as well as good oral absorption/bioavailability for the treatment of SCD. The invention described herein addresses these needs and provides additional benefits as well.

The P-selectin blocking activity of heparinoids is associated with concomitant L-selectin blocking,(4-6) and a selectin blocking agent under development as a panselectin inhibitor primarily blocks E-selectin but has approximately 100-fold less activity in blocking P-selectin and L-selectin (7). In contrast, different classes of agents such as monoclonal antibodies (1, 2) and nucleic acid aptamers (3) with precise specificity to block P-selectin have been engineered to exist. Among other benefits, this invention herein addresses the specificity for P-selectin that is unique to these polymeric carbohydrate agents.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### SUMMARY

Provided herein, inter alia, are compositions comprised of pentosan polysulfate sodium (PPS) components having superior oral bioavailability and P-selectin blocking activity for the treatment of sickle-cell disease (SCD) as well as methods for using the same.

Accordingly, in some aspects, provided herein are compositions comprising an isolated pentosan polysulfate sodium (PPS) fraction wherein the composition has (a) improved or comparable P-selectin blocking activity, (b) improved bioavailability, and (c) no greater anti-coagulant activity relative to unfractionated PPS. In some embodiments, unfractionated PPS has an average molecular weight range of 1,221-7,681 Da.

In other aspects, provided herein are compositions comprising an isolated pentosan polysulfate sodium (PPS) fraction having the same properties as a composition produced by (a) solubilizing PPS in an aqueous solution including but not limited to water; (b) adding an organic solvent in a stepwise manner to the solubilized PPS until the total concentration of the organic solvent is at least about 50% by volume; and (c) isolating a precipitated PPS fraction. In some embodiments, the organic solvent is added in a stepwise manner to the solubilized PPS until the total concentration of the organic solvent is at least about 38% by volume. In some embodiments, steps (b) and (c) of claim 3 are repeated using progressively increasing concentrations of organic solvent comprising at least about 43%, 46%, 48%, and/or 50% by volume. In some embodiments of any of the embodiments described herein, the stepwise manner comprises removing the supernatant and re-solubilizing the precipitated PPS fraction. In some embodiments of any of the embodiments described herein, the properties are (a) improved p-selectin blocking activity, (b) improved bioavailability, and (c) no greater anti-coagulant activity relative to unfractionated PPS. In some embodiments, unfractionated PPS has an average molecular weight range of 1,221-7,681 Da. In some embodiments, the composition is produced by (1) solubilizing PPS in an aqueous solution including but not limited to water; (2) adding an organic solvent in a stepwise manner to the solubilized PPS until the total concentration of the organic solvent is at least about 50% by volume; and (3) isolating a precipitated PPS fraction. This aqueous solution may contain salts, such as sodium chloride, co-solvents, surfactants, or other additives. In some embodiments, the organic solvent is added in a stepwise manner to the solubilized PPS until the total concentration of the organic solvent is at least about 38% by volume. In some embodiments, wherein steps (2) and (3) are repeated using progressively increasing concentrations of organic solvent comprising at least about 43%, 46%, 48%, and/or 50% by volume. In some embodiments of any of the embodiments described herein, the stepwise manner comprises removing the supernatant and re-solubilizing the precipitated PPS fraction. In some embodiments of any of the embodiments described herein, the organic solvent is selected from the group consisting of methanol, ethanol, propanol, and butanol. In some embodiments of any of the embodiments described herein, the organic solvent is methanol. In some embodiments of any of the embodiments described herein, the isolated PPS fraction has a weight average molecular weight (Mw) of between about 3761-4832 Da. In some embodiments, the isolated PPS fraction has a weight average molecular weight (Mw) of about 4274 Da. In some embodiments of any of the embodiments described herein, the isolated PPS fraction has a polydispersity index of between about 1.237-1.142 Mw/Mn. In some embodiments, the isolated PPS fraction has a polydispersity index of about 1.167 Mw/Mn. In some embodiments of any of the embodiments described herein, the composition exhibits reduced E-selectin and L-selectin blocking activity compared to P-selectin blocking activity. In some embodiments, the composition exhibits less than 5% E-selectin blocking activity. In some embodiments, the composition exhibits less than 2% L-selectin blocking activity.

In further aspects, provided herein are pharmaceutical compositions comprising any of the compositions disclosed herein. In some embodiments, the pharmaceutical composition further comprises one or more of an anionic surfactant, a cationic surfactant, a zwitterionic surfactant, an organic acid, and/or an antioxidant.

In other aspects, provided herein are methods for treating sickle cell disease (SCD) in a subject in need thereof, comprising administering a clinically effective amount of any of the fractionated pentosan polysulfate sodium (PPS) compositions disclosed herein or the any of the pharmaceutical compositions disclosed herein to the subject. In some embodiments, the composition or the pharmaceutical composition is administered orally. In other embodiments the composition or the pharmaceutical composition can be administered subcutaneously, intravenously, intramuscularly, by inhalation, transdermally, topically, or by any other acceptable pharmaceutical route of administration.

In yet other aspects, provided herein are kits comprising (a) any of the isolated pentosan polysulfate sodium (PPS) fractions disclosed herein and (b) one or more pharmaceutically acceptable excipients, carriers, adjuvants or vehicles.

In still other aspects, provided herein are methods for detecting pentosan polysulfate sodium (PPS) or a PPS fraction in a biological sample, the method comprising: (a) contacting the sample with a protease; (b) extracting and precipitating the PPS or the PPS fraction in the sample; (c) contacting the sample with an antibody which binds to PPS or the PPS fraction, wherein the antibody is directly or indirectly capable of detection; and (d) detecting the antibody, thereby detecting the presence of PPS or the PPS fraction in the biological sample. In some embodiments, the biological sample is blood. In some embodiments, the biological sample is serum or plasma. In some embodiments of any of the embodiments disclosed herein, the method has a Lower Limit of Detection (LLOD) (2× signal/background) of between about 0.5 ng/mL to about 10 ng/mL. In some embodiments of any of the embodiments disclosed herein, the antibody which binds to PPS or the PPS fraction is used in an ELISA assay. In some embodiments of any of the embodiments disclosed herein, the PPS is extracted and precipitated with chloroform and ammonium acetate. In some embodiments of any of the embodiments disclosed herein, step (b) is performed before step (a). In some embodiments of any of the embodiments disclosed herein, the PPS fraction is any one of the PPS fractions disclosed herein.

Each of the aspects and embodiments described herein are capable of being used together, unless excluded either explicitly or clearly from the context of the embodiment or aspect.

### BRIEF DESCRIPTION OF THE DRAWINGS

FIG. 1 depicts the P-selectin blocking Specific Activity of PPS and PPS fractions 3-9. Y-axis is IC50 of P-selectin blocking activity (nmol/mL). IC50 of PPS is shown as horizontal line. PPS fractions are designated on X-axis.

FIG. 2 depicts mean non-volatile cumulative urinary excretion radioactivity for PPS, Fraction 5, and Fraction 7 through 96 hr. The data points are mean urinary radioactivity from triplicate measurements for each test item from which the volatile radioactivity of tritiated water was removed. As shown, the fractions have greater urinary excretion through 96 hours in comparison to unfractionated PPS.

FIG. 3 depicts mean non-volatile radioactivity concentrations of PPS, Fraction 5, and Fraction 7 in plasma samples. The data points are mean plasma radioactivity from triplicate measurements for each test item from which the volatile radioactivity of tritiated water was removed. As shown, the fractions have greater oral BA than PPS and remain bioavailable even after 48 hours in comparison to unfractionated PPS.

FIG. 4 depicts blocking specificity of unfractionated PPS as well as Fractions 5 and 7 for P-selectin, E-selectin, and L-selectin. As shown, unfractionated PPS, Fraction 5, and Fraction 7 are highly specific with respect to blocking P-selectin compared to the ability to block E-selectin or L-selectin over a range of concentrations.

FIG. 5 depicts signal over noise for a modified ELISA assay used to detect the presence and quantity of PPS in either serum or plasma from humans or rats.

FIG. 6 depicts signal over noise for a modified ELISA assay to detect either PPS or Fraction 5 in human, monkey, or rat serum pretreated with protease.

FIG. 7 depicts signal over noise for a modified ELISA assay to detect PPS in human or rat serum pretreated with chondroitinase.

FIG. 8 depicts signal over noise for a modified ELISA assay to detect PPS in human or rat serum. Prior to running the assay, PPS was extracted using chloroform and then precipitated with ammonium acetate and ethanol.

### DETAILED DESCRIPTION

The invention described herein provides, inter alia, isolated and/or purified fractions of pentosan polysulfate sodium (PPS) having improved P-selectin blocking activity, improved bioavailability, less heterogeneity, and no greater anti-coagulant activity relative to unfractionated PPS. In contrast to unfractionated PPS, which, according to the distributor has only a 3%-6% oral bioavailability (BA), certain PPS fractions provided herein exhibit substantially improved oral BAs. As such, the compositions described herein are beneficial for the treatment of sickle-cell disease (SCD), as PPS has been shown to improve microvascular blood flow in patients with SCD via blocking of P-selectin.

### I. Definitions

The “Polydispersity Index” (PDI) of a polymer is defined as the ratio of the weight average molecular weight of the polymer to the number average molecular weight of the polymer (Mw/Mn). A PDI of 1.2 or less indicates that the distribution is monodisperse.

By “purified” and “isolated” is meant, when referring to a PPS fraction, that the fraction has a PDI of less than or equal to 1.25.

In some embodiments, the phrase “PPS having an average molecular weight range of 1,221-7,681 Da” refers to unfractionated and/or commercially available PPS although the actual MW range of unfractionated PPS is much broader.

By “improved P-selectin blocking activity” is meant, when referring to a PPS fraction, that a specific isolated fraction increases the ability to block P-selectin molecular adhesion to immobilized Sialyl Lewis A (“sLeA”) in vitro relative to a comparison PPS preparation. As noted herein, in some embodiments, the PPS fraction of the present invention can exhibit improved P-selectin blocking activity relative to unfractionated PPS or a PPS preparation having an average molecular weight range of between about 1,221-7,681 Da. In some embodiments a specific molecular weight range also increases the ability to block adhesion of U937 cells, HL-60 cells, sickle erythrocytes, and/or leukocytes to immobilized P-selectin relative to a comparison PPS preparation.

By “no greater anticoagulation activity” is meant, when referring to a PPS fraction, that this specific fraction exhibits no significantly greater anticoagulant activity relative to an unfractionated PPS preparation. In some embodiments, the PPS fraction of the present invention exhibits no greater anticoagulation activity relative to unfractionated PPS having an average molecular weight range of between about 1,221-7,681 Da.

By “improved bioavailability” is meant, when referring to a PPS fraction, that a specific fraction improves the AUC, Cₘₐₓ, plasma concentration, and/or total cumulative urinary excretion of PPS when orally delivered relative to an unfractionated PPS preparation. For a single administration, the Cₘₐₓ is the maximum plasma concentration, the AUC is the mathematically integrated area under the plasma concentration-time curve, and the cumulative urinary excretion is the amount of unlabeled or labeled (e.g., radioactively labeled) PPS appearing in a sample (such as, without limitation, urine or plasma) over time. As noted herein, in some embodiments, the PPS fraction of the present invention exhibits improved bioavailability relative to an unfractionated PPS preparation or a PPS preparation having an average molecular weight range between about 1,221-7,681 Da.

By “improving the duration of action” is meant either that a specific PPS fraction increases T_{1/2} or the duration of the desired pharmacological effect. “T_{1/2},” as used herein, is the half-life or half-lives.

The “number average molecular weight” (Mn) refers to the total weight of the sample divided by the number of molecules in the sample and is the first moment about the mean; it can be measured by gel permeation chromatography and refractive index and emphasizes to the lowest mw portion of a sample.

The “weight average molecular weight” (Mw) is the ratio of the second to the first moment about the mean can be determined by, for example, gel permeation chromatography and light scattering and is the average molecular weight closest to the center of a given chromatographic peak.

The “Z average molecular weight” (Mz) determined by viscosity reflects the average molecular weight closest to the highest molecular weight portion of the sample. In some embodiments it refers to the ratio of the third to the second moment about the mean and is important for skewed distributions and determined from viscosity.

By “improved microvascular blood flow” is meant, when referring to a PPS fraction, that a specific fraction increases the microvascular blood flow in a sickle cell mouse chimera model system when orally delivered relative to, for example, a comparison PPS preparation or blood flow in an untreated sickle cell mouse chimera system. As noted herein, in some embodiments, the PPS fraction of the present invention exhibits protection against induced microvascular blood flow stoppage relative to an unfractionated PPS preparation or a PPS preparation having an average molecular weight range of between about 1,221-7,681 Da. Microvascular blood flow also can be measured by laser Doppler velocimetry or any of several other non-invasive methods including Computer-Assisted Intravital Microscopy (CAIM), laser speckle contrast imaging, EndoPAT, contrast-enhanced ultrasound microbubble flow imaging, fingertip temperature rebound, and orthogonal polarization spectral imaging.

“Sample” or “biological sample” refers to a sample from a human, animal, placebo, or research sample, e.g., a cell, tissue, organ, fluid, gas, aerosol, slurry, colloid, or coagulated material. The “sample” may be tested in vivo, e.g., without removal from the human or animal, ex vivo, e.g., after removal from the human or animal, or in vitro, e.g., in a nonliving environment. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

A “subject” can be a vertebrate, a mammal, or a human. Mammals include, but are not limited to, farm animals, sport animals, pets, primates, mice and rats. In one aspect, a subject is a human.

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

### II. Compositions of the Invention

A. Pentosan Polysulfate Sodium (PPS)

Pentosan polysulfate sodium (PPS) is a high molecular weight sulfated polysaccharide that has structural and functional similarities to heparin. Unfractionated PPS is a heterogeneous mixture prepared by sulfation of polymeric xylose molecules, typically extracted from the pulp of the beech tree Fagus sylvatica. In one embodiment, unfractionated PPS has an average molecular weight range of between about 1,221-7,681 Da and as an oral agent has 3%-6% bioavailability (BA) and a half-life of 4.8 hr. Elmiron® (Janssen Pharmaceuticals, Inc.), which is supplied in white opaque hard gelatin capsules containing 100 mg PPS as an active ingredient, which is blended with microcrystalline cellulose and magnesium stearate as pharmaceutical excipients for oral use in the treatment of interstitial cystitis. In use for over eighteen years, a very large safety database for oral PPS in humans has been established. In addition, other forms of PPS have been used topically, orally, and by injection in European countries for a variety of diseases.

In some embodiments, unfractionated PPS has an average molecular weight range of between about 500-10,000 Da, such as between any of about 600-9000 Da, 700-8500 Da, 800-8000 Da, 900-9600 Da, 1000-9000 Da, 500-9500 Da, 500-9000 Da, 500-8900 Da, 500-8800 Da, 500-8700 Da, 500-8600 Da, 500-8500 Da, 500-8400 Da, 500-8300 Da, 500-8200 Da, 500-8100 Da, 500-8000 Da, 500-7900 Da, 500-7800 Da, 500-7700 Da, 500-7600 Da 600-7500 Da, 700-7400 Da, 800-7300 Da, 900-7200 Da, 1000-7100 Da, 1100-7000 Da, 1200-6900 Da, or 1500-6500 Da. In other embodiments, unfractionated PPS has an average molecular weight range of 1,221-7,681 Da. In another embodiment, unfractionated PPS has an average molecular weight range of between about any of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% below or above the range of 1,221-7,681, respectively.

Although PPS was originally developed as an antithrombotic, it was found in vitro to have only one-tenth the anticoagulant effect of unfractionated heparin on a gravimetric basis as measured by the activated Partial Thromboplastin Time (APIT) test. However, the in vivo anticoagulant activity of PPS was sufficiently greater that the APTT test could be used as a clinical measure of PPS activity. Unlike the anticoagulant effects of heparin, which are derived from enhancing the activities of anti-thrombin (AT; also known as anti-thrombin-III or AT-III) and heparin cofactor II (HC-II), certain anticoagulant activities of PPS are independent of these heparin cofactors partly as a result of its direct binding to thrombin and to factor VIIIa. These interactions of unfractionated PPS with the coagulation system provide an appreciation of its hemorrhagic risks, as well as a basis for coagulation tests useful for assessing anticoagulant activities of various PPS components.

In a Phase I study of the safety and activity of first generation (unfractionated) PPS, Elmiron® was administered as single 100 mg, 200 mg, and 300 mg oral doses separated by at least one week to five patients with sickle cell disease and as a single 300 mg dose to two healthy control subjects. There were no serious drug related drug effects, abnormalities of laboratory tests that monitor organ-specific toxicity, clinical bleeding, or abnormalities of coagulation laboratory tests. No pharmacokinetic (PK) results were obtained, for lack of a reliable assay. A beneficial pharmacodynamic (PD) effect was measured using laser Doppler velocimeter (LDV) to noninvasively monitor blood flow in the microvasculature. With LDV a laser signal from the transmitting fiber of a probe attached to the skin is directed to subcutaneous tissues, and blood flow is determined by the intensity of the signal reflected off moving red blood cells in subcutaneous capillaries back to the receiving fiber of the probe. The sufficiency of microvascular blood flow is determined most accurately by responses of signal intensity to perturbations of flow. One well-established perturbation system is the characterization of hyperemic flow following a period of flow occlusion (post-obstructive reactive hyperemia; PORH). In this PORH assessment hyperemic blood flow to tissue that had been temporarily deprived of flow achieves higher flow rates and requires a shorter payback period in subjects with normal microvascular flow, compared to patients having impaired microvascular flow in whom payback flow achieves lesser flow rates and takes longer. A different flow perturbation that has been used in LDV is thermal induction of blood flow, for which the probe contains a thermal transducer and sufficiency of microvascular blood flow is determined by the peak flow attained during a period of thermal stimulation. This perturbation was used to demonstrate improved sickle cell blood flow in response to treatment with a vasodilator or with hydroxyurea.

In the study of Elmiron® administered as a single 300 mg oral dose to five sickle cell patients, microvascular blood flow increased at 1 hr, 3 hr, 6 hr, and 8 hr after dosing, as reflected by shortened times to half peak flow compared to pretreatment values. As a result of the single best responder creating large variance in the results, these changes were not significant although there was a substantive trend. When that best responder was excluded from analysis, the improvement in half times to peak flow was statistically significant. The times recorded after PPS treatment of these patients were in the same range as the times of the two healthy control subjects before and after treatment with 300 mg PPS. This small sampling of patients did not reveal detectable improvements with doses of 100 mg or 200 mg or in peak flow measurements at any of the three doses.

An exploratory Phase II study of first generation PPS (Elmiron®) administered orally as 300 mg daily doses was intended to demonstrate activity of PPS in sickle cell disease using as endpoints improvement in microvascular flow measured by LDV, improvements in one or more markers of vascular endothelial injury, or a trend in reduction of pain measured by a daily pain diary or frequency of pain crisis (α=0.10) in a study of 80 patients. This multicenter, placebo-controlled, randomized, double blind study included a 3-week lead-in period for baseline data collection, a 12-week period on drug or placebo, and a 4-week run down period after dosing was completed. This study was not designed or powered to demonstrate a significant reduction in pain (α=0.05). Per protocol analysis of the collected data revealed that in seven patients who had taken active drug reliably for at least eight weeks there was a highly significant reduction in a marker of vascular injury, soluble VCAM-1 (p=0.01). In addition, PORH changes measured by LDV revealed trends toward improved microvascular blood flow as reflected in shorter times to half peak flow (p=NS).

While these results demonstrate PD effects of first generation PPS in patients with sickle cell disease, this product is not optimal for the long-term treatment of this disorder because of its marginal bioavailability and limited half-life of approximately 4.8 hours when given orally. Reportedly, more than 94% of the medication is excreted, intact, in feces without providing any beneficial effect. Typical variance in oral absorption of drugs has the unacceptable consequence for drugs with low bioavailability that substantial numbers of patients have no absorption at all.

B. Isolated PPS Fractions

Provided herein are compositions comprising an isolated pentosan polysulfate sodium (PPS) fraction wherein the composition has improved P-selectin blocking activity, improved bioavailability, and/or no greater anti-coagulant activity relative to unfractionated PPS. Commercially available and unfractionated sources of PPS have reported molecular weights in the range of 4-6 kDa but in actuality are broader. PPS fractions may be isolated and purified using any suitable means known in the art including, without limitation, selective precipitation with an organic solvent (such as, methanol or ethanol precipitation), size exclusion chromatography, or ion exchange chromatography.

In some aspects, the compositions comprising an isolated PPS fraction as provided herein have properties similar to compositions produced by the following process. PPS can be solubilized in an aqueous solution including but not limited to water to a concentration of any of about 5%, 10%, 15%, 20%, or 25% or more, inclusive of any percentages falling within these values. In some embodiments, an organic solvent is added to the solubilized PPS solution in order to precipitate the PPS by molecular weight. While higher molecular weight species or more highly charged species will be the first to precipitate, as the concentration of organic solvent in the PPS solution increases, progressively lower molecular weight or less charged species will also precipitate out of the solution.

The solvent used in the process can be any organic solvent including, without limitation, organic alcohols such as methanol, ethanol, propanol, butanol, pentanol, or isopropyl alcohol. The process also encompasses the stepwise addition of multiple concentrations of an organic solvent to cause the precipitation of a specific PPS fraction. For example, in one embodiment, an organic solvent (such as methanol) is added to the solubilized PPS solution in a dropwise manner until the concentration of the organic solvent reaches any of about 25%, 26%, 27%, 28%, 29%, 30%, 31%, 32%, 33%, 34%, 35%, 36%, 37%, or 38% by volume. At this point, the solution can be centrifuged and the supernatant removed. The precipitate remaining following centrifugation and removal of supernatant can then be re-solubilized and assessed for properties such as molecular weight, P-selectin blocking activity, bioavailability, and/or anti-coagulant activity using methods known in the art or described in the Examples below.

Moreover, the supernatant from the initial fractionation/precipitation described above can be further fractionated by the addition of progressively increasing concentrations of organic solvent (such as methanol) followed by centrifugation, removal of the supernatant, and re-solubilization of the precipitate. This process may be repeated any number of times using any concentration of organic solvent. For example, the concentration of organic solvent sufficient to cause PPS precipitation and subsequent isolation of a PPS fraction can be any of 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75% or more. In one embodiment, precipitated PPS fractions are isolated and collected as described above and the supernatant is subjected to further addition of concentrations of organic solvent (such as methanol) of 38%, 43%, 46%, 48%, 50%, 53%, and 56% by volume. In some embodiments, the composition comprising an isolated PPS fraction improved P-selectin blocking activity, improved bioavailability, and/or reduced anti-coagulant activity relative to unfractionated PPS is the fraction isolated using 50% organic solvent (such as methanol) by volume.

Further information related to fractionation of sulfated polymers can be found in International Patent Application Publication No. WO2010000013, the disclosure of which is incorporated by reference herein in its entirety.

The isolated PPS fractions described herein can have a weight average molecular weight (Mw) of between about 2000-7000 Da, such as between about any of 2100-6900 Da, 2200-6800 Da, 2300-6700 Da, 2400-6600 Da, 2500-6500 Da, 2600-6400 Da, 2700-6300 Da, 2800-6200 Da, 2900-6100 Da, 3000-6000 Da, 3100-5900 Da, 3200-5800 Da, 3300-5700 Da, 3400-5600 Da, 3500-5500 Da, 3600-5400 Da, 3700-5300 Da, 3800-5000 Da, 3850-4750 Da, 3900-4700 Da, 3950-4650 Da, 4000-4600 Da, 4050-4550 Da, 4100-4500 Da, 4150-4450 Da, 4200-4400 Da, or 4250-4350 Da. In another embodiment, the PPS fractions described herein can have a Mw of about 4260 Da, 4261 Da, 4262 Da, 4263 Da, 4264 Da, 4265 Da, 4266 Da, 4267 Da, 4268 Da, 4269 Da, 4270 Da, 4271 Da, 4272 Da, 4273 Da, 4274 Da, 4275 Da, 4276 Da, 4277 Da, 4278 Da, 4279 Da, 4280 Da, 4281 Da, 4282 Da, 4283 Da, 4284 Da, 4285 Da, 4286 Da, 4287 Da, 4288 Da, or 4289 Da. In another embodiment, the isolated PPS fractions described herein can have a Mw of between about 3761-4832 Da

The isolated PPS fractions described herein can have a polydispersity index (PDI) of between about 0.5-1.5 Mw/Mn, such as any of about 0.6-1.4 Mw/Mn, 0.7-1.3 Mw/Mn, 0.75-1.25 Mw/Mn, 0.8-1.24 Mw/Mn, 1.145-1.235 Mw/Mn, 1.15-1.23 Mw/Mn, 1.155-1.225 Mw/Mn, 1.16-1.22 Mw/Mn, 1.165-1.215 Mw/Mn, 1.17-1.21 Mw/Mn, 1.175-1.205 Mw/Mn, 1.18-1.2 Mw/Mn, or 1.185-1.195 Mw/Mn. In another embodiment, the PPS fractions described herein can have a polydispersity index of any of about 1.16 Mw/Mn, 1.161 Mw/Mn, 1.162 Mw/Mn, 1.163 Mw/Mn, 1.164 Mw/Mn, 1.165 Mw/Mn, 1.166 Mw/Mn, 1.167 Mw/Mn, 1.168 Mw/Mn, 1.169 Mw/Mn, 1.170 Mw/Mn, 1.171 Mw/Mn, 1.172 Mw/Mn, 1.173 Mw/Mn, 1.174 Mw/Mn, 1.175 Mw/Mn, 1.176 Mw/Mn, 1.177 Mw/Mn, 1.178 Mw/Mn, 1.179 Mw/Mn, 1.180 Mw/Mn, 1.181 Mw/Mn, 1.182 Mw/Mn, 1.183 Mw/Mn, 1.184 Mw/Mn, 1.185 Mw/Mn, 1.186 Mw/Mn, 1.187 Mw/Mn, 1.188 Mw/Mn, 1.189 Mw/Mn, 1.19 Mw/Mn, 1.191 Mw/Mn, 1.192 Mw/Mn, 1.193 Mw/Mn, 1.194 Mw/Mn, 1.195 Mw/Mn, 1.196 Mw/Mn, 1.197 Mw/Mn, 1.198 Mw/Mn, 1.199 Mw/Mn, 1.2 Mw/Mn, 1.21 Mw/Mn, 1.22 Mw/Mn, 1.23 Mw/Mn, 1.24 Mw/Mn, or 1.25 Mw/Mn. In another embodiment, the isolated PPS fractions described herein can have a PDI of between about 1.142-1.237 Mw/Mn.

In some embodiments, the fractionated PPS compositions of the present invention have improved or comparable P-selectin blocking activity compared to unfractionated PPS. In some embodiments, the fractionated PPS compositions of the present invention have identical blocking activity compared to unfractionated PPS. In still further embodiments, the fractionated PPS compositions of the present invention have any of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, or more improved P-selectin blocking activity compared to unfractionated PPS, inclusive of values falling in between these percentages.

In other embodiments, the fractionated PPS compositions of the present invention do not exhibit blocking activity towards either E-selectin or L-selectin or exhibit reduced blocking activity towards either E-selectin or L-selectin. In one embodiment, the fractionated PPS compositions of the present invention exhibit less than about 10% blocking activity towards E-selectin, such as less than about 9%, 8%, 7%, 6%, 5%, 4%, 3%, 2%, or 1% blocking activity towards E-selectin. In another embodiment, the fractionated PPS compositions of the present invention exhibit less than about 3% blocking activity towards L-selectin, such as less than about 2% or 1% blocking activity towards L-selectin.

In other embodiments, the fractionated PPS compositions of the present invention have improved bioavailability compared to unfractionated PPS. In some embodiments, the fractionated PPS compositions of the present invention have any of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, or more improved bioavailability compared to unfractionated PPS.

In further embodiments, the fractionated PPS compositions of the present invention have no greater anti-coagulant activity relative to unfractionated PPS. In one embodiment, the unfractionated PPS compositions of the present invention have identical anti-coagulant activity relative to unfractionated PPS. In another embodiment, the unfractionated PPS compositions of the present invention have less anti-coagulant activity relative to unfractionated PPS, such as any of about 0.5, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15% or greater less anti-coagulant activity relative to unfractionated PPS inclusive of values falling in between these percentages. In further embodiments, the fractionated PPS compositions of the present invention have no greater anti-coagulant activity relative to unfractionated PPS such as any of no greater than 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% anti-coagulant activity relative to unfractionated PPS inclusive of values falling in between these percentages

C. Pharmaceutical Compositions

Provided herein are pharmaceutical formulations or compositions comprising any of the isolated pentosan polysulfate sodium (PPS) fractions described herein. The term “pharmaceutical” as used herein refers to a chemical substance intended for use in the cure, treatment, or prevention of disease and which is subject to an approval process by the U.S. Food and Drug Administration (or a non-U.S. equivalent thereof) as a prescription or over-the-counter drug product. Details on techniques for formulation and administration of such compositions may be found in Remington, The Science and Practice of Pharmacy 21st Edition (Mack Publishing Co., Easton, Pa.) and Nielloud and Marti-Mestres, Pharmaceutical Emulsions and Suspensions: 2nd Edition (Marcel Dekker, Inc, New York).

For the purposes of this disclosure, the pharmaceutical compositions may be administered by a variety of means including orally, parenterally, by inhalation spray, topically, by transdermal, ocular, or rectally in formulations containing pharmaceutically acceptable carriers, adjuvants and vehicles. The term parenteral as used here includes but is not limited to subcutaneous, intravenous, intramuscular, intra-arterial, intradermal, intrathecal and epidural injections with a variety of infusion techniques, and long-term injectables, or implants. Intra-arterial and intravenous injection as used herein includes administration through catheters. Administration via intracoronary stents and intracoronary reservoirs is also contemplated. The term oral as used herein includes, but is not limited to oral ingestion, or delivery by a sublingual or buccal route. Oral administration includes fluid drinks, energy bars, as well as pill formulations.

Pharmaceutical compositions may be in any form suitable for the intended method of administration. When used for oral use for example, capsules, soft gelatin capsules, sachets or stickpacks, tablets, troches, lozenges, aqueous or oil suspensions or solutions, dispersible powders or granules, emulsions, ointments, creams, gels, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents, small organic acids or buffers, such as citrate, malate, fumarate, maleate, tartrate, and others, and preserving agents, in order to provide a palatable preparation. Tablets comprised of a drug compound in admixture with non-toxic pharmaceutically acceptable excipients that are suitable for manufacture of tablets are acceptable. These excipients may be, for example, inert diluents, such as calcium or sodium carbonate, lactose, calcium or sodium phosphate, microcrystalline cellulose, maltodextrin, mannitol, and others; granulating, binding, and disintegrating agents, such as maize starch, or alginic acid; binding agents, such as starch, gelatin or acacia; superdisintegrants such as sodium croscarmellose, cross-linked povidone, and sodium starch glycolate; and lubricating agents; such as magnesium stearate, stearic acid, sodium steearyl fumarate; such as flow or anti-tack reagents such as colloidal silica or talc. Tablets, capsules, or particulates may be uncoated, or may be coated by known techniques including enteric coating, colonic coating, or microencapsulation to delay disintegration and adsorption in the gastrointestinal tract and/or provide a sustained action over a longer period. For example, a time delay material such as glyceryl monostearate or glyceryl distearate alone or with a wax may be employed. Alternatively, controlled release matrices or coatings comprised of polymers such as polyethylene oxides, polyacrylate polymers and copolymers, polymethyl methacrylate copolymers, alkyl substituted celluloses, such as hydroxypropylcellulose, hydroxypropylmethylcellulose, croscarmellose, methyl cellulose, hydroxyethylcellulose, polyvinyl alcohols, poloxamers, alginates, polyvinylpyrrolidone, ethyl cellulose, pectins, xantham gum, and other pharmaceutically oral acceptable polymers found in the Inactive Ingredients Guide (IIG) published on the FDA website. Taste masking coating, aesthetic coating, and other coatings may also be used.

Swellable gastric retentive dosage forms for PPS or its fractions that may release drug by any combination of diffusion and erosion and comprised of hydrophilic swelling polymers may be used to release drug or coated particles of drug to the stomach and small intestine. Where a controlled release delivery of PPS or its fractions is desired and when colonic permeability is substantially less than that in the small intestine, a gastric retentive dosage form may be desirable. Preferred polymers for a swelling gastric retentive dosage form for PPS or its fractions are polyethylene oxides (PEO's) or hypromellose (HPMC). When mostly diffusional release is required, higher molecular weight ranges from 4,000,000 to 10,000,000 of PEO are preferred. Higher viscosity HPMC may also be used. When erosional release with swelling is needed, lower molecular weight ranges from 900,000 to 4,000,000 are preferred. These aspects are described in U.S. Pat. Nos. 6,635,280 and 7,976,870, which are incorporated by reference. The swelling dosage form. Gastric retentive dosage forms for enteric-coated particles delivered in a pulsatile fashion are descried in US Patent Application 20090028941, which is also incorporated by reference. For these swelling dosage forms optimal bioavailability is achieved when given with food, and the best timing is often with the evening meal. In the case where it is preferable to deliver the PPS or its fraction directly to the small intestine, coated or enteric-coated particles of drug or drug and enhancers and in both cases often with other excipients are dispersed in the erodible, hydrophilic polymeric matrix and released by erosion. When an enhancer is needed, each particle or group of particles that are released together from the matrix must contain sufficient enhancer(s) to affect the intestinal membrane permeability or to enhance the bioavailability of PPS or its fractions. The duration of this controlled release delivery would be short to moderate, and typically from 1 to 8 hours, and preferably from 2 to 6 hours. As well as having a drug-containing matrix layer, the dosage form may have a second swelling layer that is comprised of a high molecular weight swelling polymer, preferably high molecular weight PEO or HPMC. This second layer, designed to aid retention in the stomach, may also contain a gas generating agent such as sodium bicarbonate with or without a small organic acid such as citric acid, maleic acid, fumaric acid and so on. The dosage form should swell to a size to promote gastric retention in the fed stomach where the pyloric sphincter is contracted.

Any dosage form may be coated with an aesthetic coating such as Opadry that has minimal effect on release from the dosage form. Examples of enhancers are bile salts such as sodium cholate, sodium taurocholate, sodium deoxycholate, sodium glycocholate, ursodeoxycholate, acyl carnitine, lauroyl carnitine, fatty acids or their salts, such as capric acid, caprylic acid, lauric acid, oleic acid, gallate esters, TPGS, lecithins, betaines, tocopherol derivatives, small organic acids such as lactic acid, citric acid, maleic acid, fumaric acid, sorbic acid, tartaric acid, malic acid, or others, Cremophor EL or RH40, Tween 80, glycerol monoleate, glycerol caprylate, lecithin, sorbitan monolaurate, and other enhancers.

Other excipients to aid dispersion or absorption of the drug including surfactants and enhancers may be incorporated into the dosage form.

Formulations for oral use may be also presented as hard gelatin or hypromellose capsules where the drug compound is mixed with an inert solid diluent, for example calcium phosphate or kaolin, or as soft gelatin capsules wherein the active ingredient is mixed with water or an oil medium, such as peanut oil, liquid paraffin or olive oil or solubilizers to form a microemulsion, SEDDS, or SMEDDS, such as Labrasol, Cremophor EL or RH, Captex 355, Capmul MCM, Peceol, lecithins, and others.

Pharmaceutical compositions may be formulated as aqueous suspensions in admixture with excipients suitable for the manufacture of aqueous-suspensions. Such excipients include a suspending agent, such as sodium carboxymethylcellulose (also known as croscarmellose), methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Preservatives are often included in liquid formulations to avoid bacterial or fungal growth.

Oil suspensions may be formulated by suspending the active ingredient in a vegetable oil, such as arachis oil, olive oil, sesame oil or coconut oil, or a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid or its derivatives or salts, tocopherol or its derivatives or salts, BHT, BHA, propyl gallate or other gallate esters, citric acid, EDTA, thiols, such as sodium sulfite, sodium metabisulfite, cysteine, monothioglycerol, and others in the IIG. While sodium metabisulfite can induce sickling in the in vitro sickle cell test at a concentration of 20 mg/ml, the greatest concentration of sodium metabisulfite possible in human blood from this dosage form would be 0.0008 times this value.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. Any surfactant listed in the IIG may be considered for use to stabilize an emulsion or suspension. The emulsion may also contain sweetening and flavoring agents and preservatives.

Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately from 0.01 mg to 1000 mg and preferably from 20 to 600 mg of active material compounded with an appropriate and convenient amount of carrier material, which may vary from about 5 to more than 99% of the total compositions. It is preferred that the pharmaceutical composition be prepared which provides easily measurable amounts for administration. Typically, an effective amount to be administered systemically is about 0.01 mg/kg to about 100 mg/kg and depends upon a number of factors including, for example, the age and weight of the subject (e.g., a mammal such as a human), the precise condition requiring treatment and its severity, the route of administration, and will ultimately be at the discretion of the attendant physician or veterinarian. It will be understood, however, that the specific dose level for any particular patient will depend on a variety of factors including the activity of the specific compound employed, the age, body weight, general health, sex and diet of the individual being treated; the time and route of administration; the rate of excretion; other drugs which have previously been administered; and the severity of the particular condition undergoing therapy, as is well understood by those skilled in the art.

As noted above, formulations of the disclosure suitable for oral administration may be presented as discrete units such as capsules, sachets or tablets each containing a predetermined amount of the active ingredient, as a powder or granules; as a solution or a suspension in an aqueous or non-aqueous liquid, or as an oil-in-water liquid emulsion or a water-in-oil liquid emulsion. The pharmaceutical compositions may also be administered as a bolus, electuary or paste.

Free flowing powders for dosage forms may be prepared by dry blending of powders, granulation whether roller compacted or slugged, high shear or fluid bed, using aqueous media or other solvents, spray drying, extruded, coated particles or other means. The drug substance, as a powder or granule, and other excipients may be screened, milled, and mixed in one or multiple steps. Mixing may occur in cube, drum, turbula, V−, or other blenders or mixers. Tests of flow are well known to persons of skill in the art to determine adequate flow and experimental designs to determine appropriate mixing parameters.

In one embodiment there is an enteric coating that is made from acrylic acid, methacrylic acid or ethacrylic acid polymers or copolymers, cellulose acetate (and its succinate and phthalate derivatives), hydroxypropyl methyl cellulose phthalate, polyvinyl acetate phthalate, hydroxyethyl ethyl cellulose phthalate, cellulose acetate tetrahydrophtalate, acrylic resin or shellac. In another embodiment the polymer is chosen from cellulose acetate phthalate (CAP; dissolves above pH 6), polyvinyl acetate phthalate (PVAP, disintegrates at pH 5), hydroxypropyl methyl cellulose phthalate (HPMCP, grade HP50 disintegrates at pH 5 and HP50 disintegrates at 5.5), methylacrylic acid copolymers (Eudragit L 100 and L12.5 disintegrate between about 6 and about 7, Eudragit L-30 and L100-55 disintegrate at pH greater than 5.5 and Eudragit S100, S12.5 and FS 30D disintegrate at pH greater than 7).

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as fluid bed or Wurster coaters, or spray or pan coating are employed to apply coatings. The coating thickness must be sufficient to ensure that the oral dosage form remains intact until the desired site of topical delivery in the intestinal tract is reached. The amount of plasticizer is optimized for each enteric coating layer and the applied amount of said polymer(s), in such a way that the mechanical properties, i.e. flexibility and hardness of the enteric coating layer(s), for instance exemplified as Vickers hardness, are adjusted so that if a tablet is desired the acid resistance of the pellets covered with enteric coating layer(s) does not decrease significantly during compression of pellets into tablets. The amount of plasticizer is usually above 5% by weight of the enteric coating layer polymer(s), (In one embodiment the amount of plasticizer is 15-50%. In another embodiment the amount of plasticizer is 20-50%). The maximum thickness of the applied enteric coating is normally only limited by processing conditions and the desired dissolution profile.

Colorants, surfactants, anti-adhesion agents, antifoaming agents, lubricants (e.g., carnuba wax or PEG) and other additives may be added to the coatings besides plasticizers to solubilize or disperse the coating material, and to improve coating performance and the coated product. To accelerate the dissolution of the enteric coat, a half-thickness, double coat of enteric polymer (for instance, Eudragit L30 D-55) may be applied, and the inner enteric coat may have a buffer up to pH 6.0 in the presence of 10% citric acid, followed by a final layer of standard Eudragit L 30 D-55.

A tablet may be made by compression or molding, optionally with one or more accessory ingredients. Compressed tablets may be prepared by compressing in a suitable tablet press the active ingredient in a free flowing form such as a powder or granules, optionally mixed with a binder (e.g., povidone, gelatin, hydroxypropyl ethyl cellulose), lubricant, inert diluent, preservative, disintegrant (e.g., sodium starch glycolate, cross-linked povidone, cross-linked sodium carboxymethyl cellulose) surface active or dispersing agent or other excipients. Molded tablets may be made by extrusion a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric or colonic coating to provide release in parts of the gut other than the stomach.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; quick dissolving sublingual or oral; tablets or films; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

As used herein, pharmaceutically acceptable salts include, but are not limited to: acetate, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris (hydoxymethyl) aminomethane. Additional pharmaceutically acceptable salts are known to those skilled in the art.

An effective amount for a particular patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

An effective amount may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of pharmaceutical composition. Where there is more than one administration of a pharmaceutical composition in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Philadelphia, Pa.; Chabner and Longo (eds.) (2001) Cancer Chemotherapy and Biotherapy, Lippincott, Williams & Wilkins, Philadelphia, Pa.).

### III. Methods of the Invention

A. Methods for Isolating PPS Fractions

Provided herein are methods for isolating pentosan polysulfate sodium (PPS) fractions with (a) improved P-selectin blocking activity, (b) improved bioavailability, (c) no greater anti-coagulant activity, and (d) less heterogeneity relative to unfractionated PPS.

In some embodiments the method encompasses solubilizing PPS in an aqueous solution including but not limited to water, adding an organic solvent in a stepwise manner to the solubilized PPS until the total concentration of the organic solvent is at least about 50% by volume, and isolating a precipitated PPS fraction. PPS can be solubilized in an aqueous solution including but not limited to water to a concentration of any of about 5%, 10%, 15%, 20%, or 25% or more, inclusive of any percentages falling within these values. In some embodiments, the pH is adjusted to greater than neutral pH, preferably to greater than pH 8, and more preferably around pH 9. In some embodiments, an organic solvent is added to the solubilized PPS solution in order to precipitate the PPS by molecular weight. While higher molecular weight and more highly charged species will be the first to precipitate, as the concentration of organic solvent in the PPS solution increases, progressively lower molecular weight and less highly charged species will also precipitate out of the solution.

The solvent used in the methods described herein can be any organic solvent including, without limitation, organic alcohols such as methanol, ethanol, propanol, butanol, pentanol, or isopropyl alcohol. The instant methods also encompass the stepwise addition of multiple concentrations of an organic solvent to cause the precipitation of a specific PPS fraction. For example, in one embodiment, an organic solvent (such as methanol) can be added to the solubilized PPS solution in a dropwise manner while stirring until the concentration of the organic solvent reaches any of about 25%, 26%, 27%, 28%, 29%, 30%, 31%, 32%, 33%, 34%, 35%, 36%, 37%, or 38% by volume. At this point, the solution can be centrifuged and the supernatant removed. The precipitate can be washed with an alcohol, preferably ethanol to remove excess aqueous solution and then dried to remove remaining solvent. The precipitate remaining following centrifugation and removal of supernatant can then be re-solubilized and assessed for properties such as molecular weight, capillary electrophoresis, P-selectin blocking activity, bioavailability, and/or reduced anti-coagulant activity using methods known in the art or described in the Examples below.

Moreover, the supernatant from the initial fractionation/precipitation described above can be further fractionated by the addition of progressively increasing concentrations of organic solvent (such as methanol) followed by centrifugation, removal of the supernatant, and re-solubilization of the precipitate. This process may be repeated any number of times using any concentration of organic solvent. For example, the concentration of organic solvent sufficient to cause PPS precipitation and subsequent isolation of a PPS fraction can be any of 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75% or more. In one embodiment, precipitated PPS fractions are isolated and collected as described above and the supernatant is subjected to further addition of concentrations of organic solvent (such as methanol) of 38%, 43%, 46%, 48%, 50%, 53%, and 56% by volume. In some embodiments, the composition comprising an isolated PPS fraction improved P-selectin blocking activity, improved bioavailability, and/or reduced anti-coagulant activity relative to unfractionated PPS is the fraction isolated using 50% organic solvent (such as methanol) by volume.

B. Methods for Treating SCD and Related Diseases

The disclosed compositions can be used to treat subjects with one or more mutations in the beta-globin gene (HBB gene). Mutations in the beta globin gene can cause sickle cell disease (SCD), beta thalassemia, or related diseases or conditions. Mutations in the beta-globin gene can be identified before or after manifestations of a disease's clinical symptoms. The isolated PPS fractions of the present invention can be administered to a subject with one or more mutations in the beta-globin gene before or after the onset of clinical symptoms. Therefore, in some embodiments, the compositions are administered to a subject that has been diagnosed with one or more mutations in the beta-globin gene, but does not yet exhibit clinical symptoms. In some embodiments, the compositions are administered to a subject that is exhibiting one or more symptoms of a disease, condition, or syndrome associated with, or caused by one or more mutations in the beta-globin gene.

Sickle cell disease (SCD) typically arises from a mutation substituting thymine for adenine in the sixth codon of the beta-chain gene of hemoglobin (i.e., GAG to GTG of the HBB gene). This mutation causes glutamate to valine substitution in position 6 of the Hb beta chain. The resulting Hb, referred to as HbS, has the physical properties of forming polymers under deoxy conditions. SCD is typically an autosomal recessive disorder. Therefore, in some embodiments, the disclosed compositions and methods are used to treat a subject homozygous for an autosomal recessive mutation in beta-chain gene of hemoglobin (i.e., homozygous for sickle cell hemoglobin (HbS)), also referred to as HbSS disease or sickle cell anemia (the most common form of SCD). Subjects homozygous for the S globin typically exhibit a severe or moderately severe phenotype and have the shortest survival of the sickle hemoglobinopathies.

Sickle cell trait or the carrier state is the heterozygous form characterized by the presence of around 40% sickle cell hemoglobin (HbS), absence of anemia, inability to concentrate urine (isosthenuria), and hematuria. Under conditions leading to hypoxia, it may become a pathologic risk factor. Accordingly, in some embodiments, the disclosed compositions and methods are used to treat a subject heterozygous for an autosomal recessive mutation in the beta-chain gene of hemoglobin (i.e., heterozygous for HbS).

Beta-thalassemias (β-thalassemias) are a group of inherited blood disorders caused by a variety of mutational mechanisms that result in a reduction or absence of synthesis of β-globin and leading to accumulation of aggregates of unpaired, insoluble α-chains that cause ineffective erythropoiesis, accelerated red cell destruction, and severe anemia. Subjects with beta-thalassemia exhibit variable phenotypes ranging from severe anemia to clinically asymptomatic individuals. The genetic mutations that cause β thalassemias are diverse and numerous. The mutations can involve single base substitutions or deletions or inserts within, near, or upstream of the β-globin gene. For example, mutations occur in the promoter regions 5′ of the β-globin genes or cause production of abnormal splice variants. Examples of the clinical thalassemia syndromes include thalassemia minor, thalassemia intermedia, and thalassemia major.

Although carriers of sickle cell trait do not suffer from SCD, individuals with one copy of HbS and one copy of a gene that codes for another abnormal variant of hemoglobin, such as HbC or beta-thalassemia, have a less severe form of the disease. For example, another specific mutation in beta-globin causes a different structural variant, hemoglobin C. Hemoglobin C (abbreviated as Hb C or HbC) is an abnormal hemoglobin in which there is a substitution of a glutamic acid residue with a lysine residue at the 6th position of the β-globin chain. A subject that is a compound heterozygote for HbS and HbC (HbSC disease) is typically characterized by symptoms of moderate clinical severity. Beta thalassemia is characterized by the reduced (β+) or absent (β^{∘}) production of normal β-globin chains. A subject that is a compound heterozygote for HbS and β+-thalassemia (HbS/β+ thalassemia) is typically characterized by symptoms of moderate clinical severity, such as HbSC disease. A subject that is a compound heterozygote for HbS and β°-thalassemia (HbS/β^{∘} thalassemia) is typically characterized by severe clinical symptoms, such as homozygous HbSS (SCA).

Another common structural variant of beta-globin is hemoglobin E or hemoglobin E (Hb E; HbE). HbE is an abnormal hemoglobin in which substitution of a glutamic acid residue with a lysine residue at the 26th position of the β-globin chain has occurred. A subject that is a compound heterozygote for HbS and HbE has HbS/HbE syndrome, which usually has a phenotype similar to HbS/β+ thalassemia.

Rare combinations of HbS with other abnormal hemoglobins include HbD Los Angeles, G-Philadelphia, HbO Arab, and others. Therefore, in some embodiments, the disclosed compositions and methods are used to treat a subject with an HbS/β^{∘} genotype, an HbS/β+ genotype, an HbSC genotype, an HbS/HbE genotype, an HbS/HbD Los Angeles genotype, an HbS/G-Philadelphia genotype, or an HbS/HbO Arab genotype.

Some of the disclosed methods include administering to the subject a second active agent, for example, vitamin supplements, nutritional supplements, anti-anxiety medication, anti-depression medication, anti-coagulants, clotting factors, anti-inflammatories, steroids such as corticosteroids, analgesic, etc. In some embodiments, the compositions are co-administered in combination with one or more additional active agents for treatment of sickle cell disease, beta-thalassemia, or a related disorder. Such additional active agents may include, but are not limited to, folic acid, penicillin or another antibiotics, preferably a quinolone or macrolide, antivirals, anti-malarial prophylactics, and analgesics to control pain crises. In some embodiments, the compositions are co-administered with one or more additional agents that increase expression of fetal hemoglobin (HbF), for example, hydroxyurea.

In subjects with SCD, or a related disorder, physiological changes in RBCs can result in a disease with the following signs: (1) hemolytic anemia; (2) vaso-occlusive crisis; and (3) multiple organ damage from microinfarcts, including heart, skeleton, spleen, and central nervous system. As such, the compositions described herein can be used to alleviate any of these common symptoms of SCD by administering a clinically effective amount of the composition to a subject exhibiting one or more of the following symptoms of SCD.

SCD is a form of hemolytic anemia. In some forms of SCD in which subjects are homozygous for HbS, red cell survival is around 10-20 days compared to normal red blood cell survival of 120 days. Approximately one third of the hemolysis occurs intravascularly, releasing free hemoglobin (plasma free hemoglobin [PFH]) and arginase into plasma. PFH has been associated with endothelial injury including scavenging nitric oxide (NO), proinflammatory stress, and coagulopathy, resulting in vasomotor instability and proliferative vasculopathy. A possible effect of this proliferative vasculopathy is the development of pulmonary hypertension in adulthood.

Vaso-occlusive crisis occurs when the circulation of blood in small vessels is obstructed by sickled red blood cells, causing ischemic injuries. This impaired blood flow is the result of adhesion of a stickier subset of SRBC to the vascular endothelium, a longer exposure of SRBC to the low oxygen tensions that induce HbS polymerization and SRBC sickling and rigidification, and physical trapping of more rigid SRBC behind the adherent SRBC. The most common complaint is of pain, and recurrent episodes may cause irreversible organ damage. One of the most severe forms is the acute chest syndrome, which occurs as a result of infarction of the lung parenchyma. Vaso-occlusive crisis can be accompanied by a pain crisis, which can occur suddenly and last several hours to several days. The pain can affect any body part. It often involves the abdomen, bones, joints, and soft tissue, and it may present as dactylitis (bilateral painful and swollen hands and/or feet in children), acute joint necrosis or avascular necrosis, or acute abdomen. With repeated episodes in the spleen, infarctions and autosplenectomy predisposing to life-threatening infection are usual. The liver also may infarct and progress to failure with time. Papillary necrosis is a common renal manifestation of vaso-occlusion, leading to isosthenuria (i.e., inability to concentrate urine) and hematuria. Severe deep pain is present in the extremities, involving long bones. Abdominal pain can be severe, resembling acute abdomen; it may result from referred pain from other sites or intra-abdominal solid organ or soft tissue infarction. Reactive ileus leads to intestinal distention and pain. Bone pain and abdominal pain may be present. The face also may be involved. Pain may be accompanied by fever, malaise, and leukocytosis.

Skeletal manifestations of SCD include, but are not limited to, infarction of bone and bone marrow, bone marrow hyperplasia compensatory to hemolytic anemia, secondary osteomyelitis, secondary growth defects, intravascular thrombosis, osteonecrosis (avascular necrosis/aseptic necrosis), degenerative bone and joint destruction, osteolysis (in acute infarction), Articular disintegration, myelosclerosis, periosteal reaction (unusual in the adult), dystrophic medullary calcification, bone-within-bone appearance, decreased density of the skull, decreased thickness of outer table of skull due to widening of diploe, hair on-end striations of the calvaria, osteoporosis sometimes leading to biconcave vertebrae, coarsening of trabeculae in long and flat bones, and pathologic fractures, bone shortening (premature epiphyseal fusion), epiphyseal deformity with cupped metaphysis, peg-in-hole defect of distal femur, and decreased height of vertebrae (short stature and kyphoscoliosis).

Renal manifestations of SCD include, but are not limited to, various functional abnormalities such as hematuria, proximal tubule dysfunction, impaired potassium excretion, and hyperkalemia, decreased glomerular filtration rate, and frank renal failure that may require dialysis or renal transplantation; and gross anatomic alterations, for example, hypertrophied kidneys, with a characteristic smooth, capsular surface.

Splenic manifestations of SCD can include, but are not limited to, one or more of enlargement, including rapid and/or painful enlargement known as splenic sequestration crisis, infarction, low pH and low oxygen tension in the sinusoids and splenic cords, functional impairment, autosplenectomy (fibrosis and shrinking of the spleen in advanced cases), immune deficiency and increased risk of sepsis.

C. Methods for Detecting PPS or Fraction Thereof in a Biological Sample

Also provided herein are methods for detecting PPS or a PPS fraction (such as any of the PPS fractions disclosed herein) in a biological sample. PPS has a reported 3%-6% absorption in humans administered 300-450 mg based on urinary excretion (Elmiron Package Insert). With the assumption of 6% absorption, a 100 mg oral dose would result in 6 mg of PPS in an average plasma volume of 3.4 L or 1.8 μg/mL.

In the PK analysis for their Elmiron NDA, Baker Norton used a Competitive Binding assay that utilizes charge based affinity to polybrene-sepharose in competition to known amounts of 125I-PPS, as described by MacGregor, et al. (1985, Thrombosis Haemostasis 53(3):411-4). Although the reported sensitivity for this method is 10 ng/mL in plasma, the FDA reviewer of the NDA commented on the lack of validation for this assay. This assay lacked the level of sensitivity to in order to be considered experimentally useful.

Polyclonal ELISA assays to detect PPS have been developed. However, the sensitivity of these assays in aqueous buffer below 200 ng/mL was lost when PPS was mixed with human or rat plasma. A further disadvantage of these assays is that the antibody, which was generated in rabbits, has finite availability.

Efforts to develop screens for the detection of PPS in blood using either a microfluidics approach or a mass spectrometric (MS) method were unsuccessful. With respect to the MS method, while PPS was detected, including in plasma, there were significant issues with multiple peaks and background mass interference.

Accordingly, provided herein is a sensitive and quantifiable assay to measure PPS or a PPS fraction (such as any of the PPS fractions provided herein) following administration to an individual. The method includes the steps of contacting the sample with a protease; extracting and precipitating the PPS or the PPS fraction in the sample; and contacting the sample with an antibody which binds to PPS or the PPS fraction, wherein the antibody is directly or indirectly capable of detection; and detecting the antibody, thereby detecting the presence of PPS or the PPS fraction in the biological sample.

The biological sample can be any sample from an individual that contains PPS or a specific fraction of PPS following administration to an animal or the individual. Thus, biological samples can include, without limitation, blood, urine, saliva, sweat, tears, semen, breast milk, or feces. In some embodiments, the biological sample is blood or products derived from blood, such as, without limitation, serum or plasma.

The method can detect the presence of PPS or a fraction thereof with a Lower Limit of Detection (LLOD) (2× signal/background) of between about 0.5 ng/mL to about 10 ng/mL. In some embodiments, the LLOD is between about 0.5 ng/mL to about 5 ng/mL, about 0.5 ng/mL to about 2.5 ng/mL, about 0.5 ng/mL to about 1.5 ng/mL, about 1 ng/mL to about 10 ng/mL, about 1 ng/mL to about 7.5 ng/mL about 1 ng/mL to about 5 ng/mL, about 1 ng/mL to about 2.5 ng/mL about 2 ng/mL to about 10 ng/mL, about 2 ng/mL to about 7.5 ng/mL, or about 2 ng/mL to about 5 ng/mL. In other embodiments, the LLOD is about 0.1 ng/mL, 0.2 ng/mL, 0.3 ng/mL, 0.4 ng/mL, 0.5 ng/mL, 0.6 ng/mL, 0.7 ng/mL, 0.8 ng/mL, 0.9 ng/mL, 1 ng/mL, 1.1 ng/mL 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2 ng/mL, 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.9 ng/mL, 3 ng/mL, 3.1 ng/mL, 3.2 ng/mL, 3.3 ng/mL, 3.4 ng/mL, 3.5 ng/mL, 3.6 ng/mL, 3.7 ng/mL, 3.8 ng/mL, 3.9 ng/mL, 4 ng/mL, 4.1 ng/mL, 4.2 ng/mL, 4.3 ng/mL, 4.4 ng/mL, 4.5 ng/mL, 4.6 ng/mL, 4.7 ng/mL, 4.8 ng/mL, 4.9 ng/mL, 5 ng/mL, 5.1 ng/mL, 5.2 ng/mL, 5.3 ng/mL, 5.4 ng/mL, 5.5 ng/mL, 5.6 ng/mL, 5.7 ng/mL, 5.8 ng/mL, 5.9 ng/mL, 6 ng/mL, 6.1 ng/mL, 6.2 ng/mL, 6.3 ng/mL, 6.4 ng/mL, 6.5 ng/mL, 6.6 ng/mL, 6.7 ng/mL, 6.8 ng/mL, 6.9 ng/mL, 7 ng/mL, 7.1 ng/mL, 7.2 ng/mL, 7.3 ng/mL, 7.4 ng/mL, 7.5 ng/mL, 7.6 ng/mL, 7.7 ng/mL, 7.8 ng/mL, 7.9 ng/mL, 8 ng/mL, 8.1 ng/mL, 8.2 ng/mL, 8.3 ng/mL, 8.4 ng/mL, 8.5 ng/mL, 8.6 ng/mL, 8.7 ng/mL, 8.8 ng/mL, 9 ng/mL, 9.1 ng/mL, 9.2 ng/mL, 9.3 ng/mL, 9.4 ng/mL, 9.5 ng/mL, 9.6 ng/mL, 9.7 ng/mL, 9.8 ng/mL, 9.9 ng/mL, 10 ng/mL, 10.5 ng/mL, 11 ng/mL, 11.5 ng/mL 12 ng/mL, 12.5 ng/mL, 13 ng/mL 13.5 ng/mL, 14 ng/mL, 14.5 ng/mL, or 15 ng/mL.

In some embodiments, the detectable antibody for use in the methods disclosed herein is produced using PPS or a fraction of PPS (such as any of the PPS fractions disclosed herein) as an immunogen according to methods that are well known in the art. The antibody can be a monoclonal antibody or a functional fragment thereof and can be directly or indirectly detectable via a label. The term “labeled,” with regard to the antibody, encompasses both direct labeling of the antibody by coupling (i.e., physically linking) a detectable substance to the antibody, as well as indirect labeling of the antibody by reactivity with another reagent that is directly labeled. Methods of indirect labeling include detection of the anti-PPS or anti-PPS fraction monoclonal antibody by using a labeled secondary antibody. Examples of directly detectable labels include enzymes (such as, without limitation, horse radish peroxidase, alkaline phosphatase, β-galactosidase, or acetylcholinesterase), prosthetic groups (for example, without limitation, streptavidin/biotin or avidin/biotin), fluorophores (such as, without limitation, FITC or Texas red), proteins (such as, without limitation, green fluorescent protein GFP) or a radioactive isotope (such as, without limitation, ³H, ³⁵S, or ¹²⁵I).

In further embodiments, PPS or a fraction of PPS can be extracted with an organic solvent (such as, but not limited to, chloroform) and then precipitated with a salt (such as, but not limited to ammonium acetate), prior to contacting with the antibody. Further information related to the extraction/precipitation step can be found in Mielke, et al., (1999, Clin Appl Thrombosis/Hemostasis, 5(4): 267-76, the disclosure of which is incorporated by reference herein).

Similarly, in other embodiments, the biological sample is contacted with a protease in order to decrease the protein content of the biological sample. Commercial sources of proteases are widely available and include, for example, pronase, which is a mixture of proteases isolated from the extracellular fluid of Streptomyces griseus with activity extending to both denatured and native proteins leading to complete or nearly complete digestion into individual amino acids. In some embodiments, any of about 50-150 Units (U), 75-125 U, 90-110 U, or 95-105 U of protease is used for every 100 μL of biological sample. In other embodiments, any of 75 U, 80 U, 85 U, 90 U, 95 U, 100 U, 105 U, 110 U, 115 U, 120 U, or 125 U (inclusive of all values falling in between these numbers) of protease is used for every 100 μL of biological sample. In other embodiments, the protease digestion is carried out between about 35° C. to about 39° C., such as any of about 35° C., 36° C., 37° C., 38° C., or 39° C. In yet other embodiments, the sample is contacted with the protease between about 1-48 hours, such as any of about 5-40 hours, about 10-30 hours, about 15-25 hours, about 18-20 hours, or any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more hours.

### IV. Kits

In addition, the present invention includes one or more kits comprising isolated pentosan polysulfate sodium (PPS) fractions, such as any of those described herein, as well as one or more pharmaceutically acceptable excipients, carriers, adjuvants or vehicles, such as any of those described herein. Other materials useful for performing the instantly described methods can also be included as part of the kit. For example, the kit can include buffers or labware necessary to obtain or store a PPS fraction derived from unfractionated PPS. Written materials describing the steps involved in the presently described methods can be included for instructing the user how to use the composition or kit. The kit may include instructions for using isolated PPS fractions and associated pharmaceutical compositions comprising the same for treating or preventing symptoms associated with sickle cell disease (SCD).

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The invention can be further understood by reference to the following examples, which are provided by way of illustration and are not meant to be limiting.

### EXAMPLES

### Example 1: Fractionation of PPS

Commercially available PPS has a molecular weight (mw) reported as ˜4-6 kDa. The breadth of P-selectin blocking activity vs. concentration of this compound is over 5 orders of magnitude (Kutlar, et al., (2012) Am J Hematol 87, 536-539 plus Online Supplemental Material), indicating that activity is provided by a diverse group of molecules, some of which provide disproportionate activity. This Example demonstrates that PPS can be fractionated into distinct entities according to different molecular weights or other properties of PPS, which can be assayed for activated partial thromboplastin time (AVIT) and/or other functional activities.

Materials and Methods

Unfractionated PPS was obtained from bene pharmaChem, GmbH (Geritsried, Germany). PPS was dissolved in H₂O to make a 10% solution. In this example 100 g of solid was dissolved in ₁₀₀ mL of total solution. The pH of this solution was adjusted to around 9.0 by the addition of NaOH. Organic solvent (in this Example, methanol) was added in a dropwise manner (while stirring) to make up a total concentration of 38% by volume. Methanol was added because high molecular weight fractions are the least soluble and adding an organic solvent such as methanol will affect their solubility. The high molecular weight fractions were the first to precipitate out of the solution. The first fraction was removed and the whole solution then centrifuged. Methanol was then added to the supernatant in the same manner to make up a total concentration of 43% by volume and the resulting precipitant (second fraction) was removed and the solution again centrifuged as described above. These steps were then repeated, increasing the concentration of methanol to 46% (Fraction 3), 48% (Fraction 4), 50% (Fraction 5), 53% (Fraction 6), 56% (Fraction 7), 60% (Fraction 8), 67% (Fraction 9), and 100% (Fraction 10). For each fraction, the precipitate was further rinsed using additional ethanol to remove as much aqueous solution as possible from the crystals.

Molecular weights of the fractions were identified according to refractive index (RI) and shown in Table I, with each fraction's activated partial thromboplastin time (APIT; see Marlar et al., (2008) One-Stage Prothrombin Time (PT) Test and Activated Partial Thromboplastin Time (APIT) Test; Approved Guideline—Second Edition, 2nd ed., pp 1-31, Clinical and Laboratory Standards Institute, Wayne, Pa.).

TABLE I
Table I

| Fraction | PPS | 1 | 2 | 3 | 4 | 5 | 6 | 7 | 8 | 9 | 10 |
|---|---|---|---|---|---|---|---|---|---|---|---|
| % MeOH | 0 | 38 | 43 | 46 | 48 | 50 | 53 | 56 | 60 | 67 | 100 |
| Mw (Da) | ~2730 | 2859 | 2510 | 6678 | 4702 | 3704 | 3001 | 2588 | 2145 | 1744 | 1221 |
| APTT (% PPS) | 100.0 | 78.7 | 158.5 | 153.7 | 115.7 | 101.3 | 96.3 | 83.0 | 74.6 | 63.9 | 40.4 |

### Example 2: Identification of Homogeneous PPS Fractions with Robust P-Selectin Blocking Activity

The purpose of this Example was to identify pentosan polysulfate sodium (PPS) fractions with P-selectin blocking specific activity at least equal to unfractionated PPS from among eight prepared by selective methanol (MeOH) precipitation (Stajic. (2010) A SULFATED POLYSACCHARIDE COMPOUND AND THE PREPARATION AND USE THEREOF, (PCT, Ed.), pp 1-91, Parnell Laboratories Pty Ltd.). Additional properties of an ideal fraction are a more homogeneous composition, described below as low polydispersity and low molecular weight (mw), which is predicted to increase oral bioavailability (BA).

Materials and Methods

The molecular weight (mw) of eight PPS fractions was reassessed using triple detection gel permeation chromatography (GPC) (Striegel, A., Yau, W. W., Kirkland, J. J., and Bly, D. D. (2009) Modern Size-Exclusion Liquid Chromatography: Practice of Gel Permeation and Gel Filtration Chromatography, 2nd Edition, 2 ed., Wiley, Hoboken) with two Jordi Sulfonated Plus Mixed Bed columns and a Jordi Sulfonated Plus 1000 Å column in series with 80/10/10 water with 1.88 g/L NaPO4/Isopropyl alcohol/Tetrahydrofuran solvent.

The molecular P-selectin blocking activities of PPS test items were determined using an assay of P-selectin binding to sialyl Lewis A (sLeA) (Kutlar, et al., (2012) Am J Hematol 87, 536-539 plus Online Supplemental Material). The test items were diluted in P-sel-Ig solutions to final concentrations ranging from 0.05 to 5000 μg/mL. After incubation and washing, P-sel-Ig bound to sLeA was quantified using goat anti-human Ig, color development, and a microplate reader. Averaged triplicate data were analyzed with Prism software to fit a nonlinear curve (Miller, J. R. (2003) GraphPad Prism Version 4.0 Step-by-Step Examples, In Nonlinear Standard Curves: RIA and ELISA, GraphPad Software, Inc., San Diego) and a 4-parameter algorithm (Plikaytis, et al., (1991) Journal of clinical microbiology 29, 1439-1446; Findlay & Dillard, (2007) The AAPS journal 9, E260-267) to determine the concentration at which P-selectin binding is inhibited by 50% (IC50) of each test item, with lower IC50 values indicating greater blocking activity). IC50 values were converted to Specific Activities (SA) using the Mw values in Table II.

Results

The original mw determined by refractive index (RI) and the three new mw determinations by triple detection gel permeation chromatography (GPC) (RI, light scattering [LS], viscometry) are shown in Table H. The number average mw (Mn) determined by GPC and RI provides information about the lowest mw portion of the sample; the weight average mw (Mw) determined by GPC LS is the average mw closest to the center of the chromatographic peak; and the Z average mw (Mz) determined by GPC viscometry reflects the larger mw portion of the sample. Mw/Mn is the polydispersity index (PDI). A PDI<1.2 reflects a monodisperse (monomolecular; homogeneous) sample. A PDI>1.2 reflects a polydisperse (polymolecular; heterogeneous) sample.

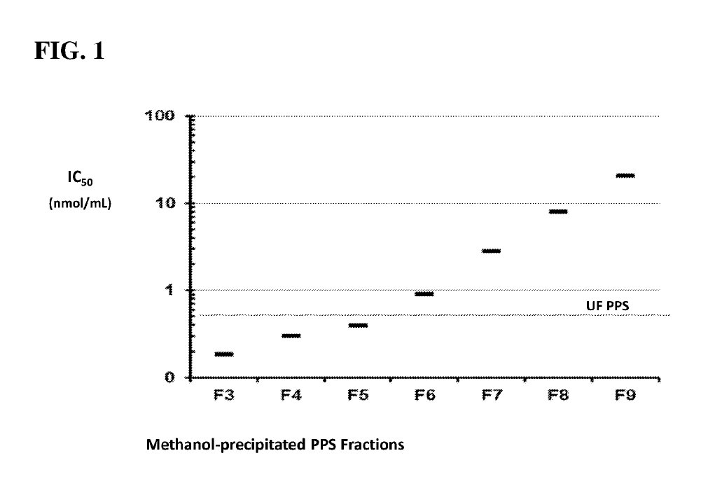

TABLE II

| Fraction | PPS | 3 | 4 | 5 | 6 | 7 | 8 | 9 | 10 |
|---|---|---|---|---|---|---|---|---|---|
| Initial Mn, weight avg. mw by | ~2730 | 6678 | 4702 | 3704 | 3001 | 2588 | 2145 | 1744 | 1221 |
| refractive index (Da) | | | | | | | | | |
| Mₙ, number avg. mw by triple | 3760 | 4594 | 3904 | 3665 | 3245 | 3034 | 2358 | 2129 | 6395 |
| detection GPC (Da) | | | | | | | | | |
| M_{w}, weight avg. mw by triple | 4994 | 6080 | 4833 | 4274 | 3760 | 3217 | 2803 | 2275 | 6336 |
| detection GPC (Da) | | | | | | | | | |
| M_{z}, Z avg. mw by triple detection | 6994 | 7681 | 5652 | 5026 | 4395 | 3457 | 3289 | 2431 | 35597 |
| GPC (Da) | | | | | | | | | |
| PDI, polydispersity index of mw | 1.329 | 1.323 | 1.238 | 1.167 | 1.141 | 1.061 | 1.190 | 1.069 | 1.774 |
| distribution (M_{w}/Mₙ) | | | | | | | | | |
| P-selectin blocking Spec. Activ. | 0.44 | 0.18 | 0.30 | 0.40 | 0.90 | 2.79 | 7.92 | 20.65 | Incalculable |
| (IC_{50;} nmol/mL)(FIG. 1) | | | | | | | | | |

Fractions 3-9 are less polydispersed than PPS, and Fractions 5-9 have PDI<1.2, which predicts more consistent absorption and pharmacodynamic activity. A PDI≤1.25 is considered here to be sufficiently homogeneous to be considered as an isolated PPS fraction. Because of their combination of lower Mw and greater homogeneity, fractions 5-9 were regarded as promising candidates after these analyses.

Regarding molecular P-selectin blocking activities of PPS test items, FIG. 1 and Table II show that only fraction 3 had Mw>PPS, fractions 3-5 had P-selectin blocking specific activity >PPS; fractions 6-9 had Mw<PPS and progressively lower P-selectin blocking specific activity (Table I; FIG. 1). Because of its greater P-selectin blocking specific activity, lower Mw, and homogeneity, fraction 5 was regarded as a potential candidate for further study. Although both Fractions 6 and 7 had lower P-selectin blocking activity than unfractionated PPS, they had less heterogeneity and lower Mw with potential or measured greater oral bioavailability than unfractionated PPS. Therefore, these candidates were also considered potential alternatives for future development.

P-selectin blocking activity also was assayed using HL60 cell adhesion to immobilized recombinant human P-selectin-Ig under flow conditions (Frangos, et al., (1988) Biotechnol Bioeng 32, 1053-1060; Matsui et al., (2002) Blood 100, 3790-3796) to determine the inhibitory effects of added compounds on P-selectin mediated cell adhesion. In these studies HL-60 cells were used because their abundant P-selectin ligand (Wilkins, et al., (1995) J Biol Chem 270, 22677-22680) provides a robust assay of P-selectin blocking. The flow of HL-60 cell suspensions was maintained at the 1 dyne/cm² shear stress of normal postcapillary venular flow (Turitto, V. T. (1982) Prog. Hemost. Thromb. 6, 139-177) and at 37° C. throughout. Cell adhesion was captured using videomicroscopy. Preliminary results on blocking P-selectin mediated cell adhesion were consistent with the molecular adhesion results detailed above. In general, the effects on decreasing the number of rolling cells and increasing the velocity of rolling cells declined directly with the mw of the fractions.

In summary, this Example identified Fraction 5 as the lowest mw PPS fraction that has P-selectin blocking specific activity ≥PPS and, on the basis of their lower mw, identified fractions 5-9 all as candidates for further bioavailability/pharmacokinetic (BA/PK) studies.

### Example 3: Identification of PPS Fractions with Increased Anticoagulant Activity

This goal of this Example was to identify and exclude from development PPS fractions that have unexpectedly high anticoagulant activity that potentially may be associated with hemorrhagic side effects. This concern is based on the observed influence of mw on the effect of PPS fractions on the APTT (see Table 1).

Materials and Methods

In vitro assays of the effects of different concentrations of the eight PPS test items (Fraction 10 was not tested) dissolved in pooled normal human plasma on the APIT, anti-Xa activity, and thrombin time (TT) were conducted according to known methods (Soria et al., (1980) Thromb Res 19, 455-463; Scully et al., (1983) Thromb Res 31, 87-97; Sie et al., (1986) Br J Haematol 64, 707-714; Wagenvoord, et al., (1988) Thromb Haemost 60, 220-225; Marlar et al., (2008) One-Stage Prothrombin Time (PT) Test and Activated Partial Thromboplastin Time (APTT) Test; Approved Guideline—Second Edition, 2nd ed., pp 1-31, Clinical and Laboratory Standards Institute, Wayne, Pa.). The concentrations of each test items were that at which PPS had been reported to have an effect (˜7.5 μg/mL for the APTT, 40 μg/mL for the anti-Xa, and 25 μg/mL for the TI), which was designated 1× for each assay. Assays were determined in duplicate.

Results

For each assay the normal range was established by replicate testing (n=20) in pooled human plasma containing no PPS. The results are shown in Table II.

TABLE III

| | Normal | | | | | | | | |
|---|---|---|---|---|---|---|---|---|---|
| Test | Range | PPS | Fr. 3 | Fr. 4 | Fr. 5 | Fr. 6 | Fr. 7 | Fr. 8 | Fr. 9 |
| APTT (Sec) | 34.0-35.2 | 102 | >300 | 87.5 | 69.5 | — | 70.9 | 61.8 | 65.9 |
| Anti-Xa (IU/mL) | <0.10 | 0.2 | 0.2 | 0.3 | 0.3 | 0.3 | 0.3 | 0.3 | 0.3 |
| TT (Sec) | 14.3-15.7 | 150 | 124 | 147 | 65.2 | 103 | 131 | 73.7 | 59.3 |

These results demonstrated that only Fraction 3 had greater anticoagulant activity than PPS. The outlier APIT result for Fraction 6 was judged to be due to a pipetting error. Assays were also run at relative concentrations 4×, 2×, 0.5× and 0.25×, and these results also showed that only Fraction 3 had greater anticoagulant effect than PPS.

### Example 4: Oral Bioavailability and Pharmacokinetics of PPS Fractions

This Example's aim was to identify a PPS fraction with greater oral BA than the parent PPS composition. Properties of PPS Fractions 5 and 7, identified in the preceding Examples, and unfractionated PPS were compared in Cynomolgus monkeys (Macaca fascicularis; Cynos). Fraction 5 was chosen because of its consistently superior properties in lower molecular weight, less polydispersity, greater P-selectin blocking activity, and no greater anticoagulant activity compared to PPS. Fraction 7 was chosen despite its much lower P-selectin blocking activity to determine whether its predicted better oral bioavailability (based on its lower molecular weight) would provide better overall pharmaceutical properties.

Materials and Methods

Fraction 5, Fraction 7, and PPS were radiolabeled using tritium gas according to standard methods and provided for testing in Cynos. The samples used were [³H]PPS-5 with a stated specific activity of 53.7 mCi/mg, [³H]PPS-7 with a stated specific activity of 65 mCi/mg, and [³H]PPS with a stated specific activity of 306 mCi/mg.

A single-dose oral pharmacokinetic study of [³H]PPS, [³H]PPS-5, and [3H]PPS-7 was conducted in Cynos to determine the plasma pharmacokinetics, excretion, and oral bioavailability (BA) of total radioactivity following a single oral dose of [³H]PPS, [³H]PPS-5, or [³H]PPS-7 to Cynos. This non-GLP study was conducted in accordance with the protocol and the testing facility Standard Operating Procedures and standard radiation safety practices. Appropriate amounts of [³H]PPS, [³H]PPS-5, or [³H]PPS-7 and the corresponding unlabeled PPS, PPS-5, or PPS-7 (as appropriate) were dissolved in the appropriate volume of water in a glass container using a stir bar on a magnetic stir plate and/or sonication. The formulations were stirred until a clear solution is obtained. Dose formulations containing isotopically diluted [³H]PPS, [³H]PPS-5, or [³H]PPS-7 were prepared at a total concentration of 0.5 mg/mL and a radioconcentration of approximately 60-65 μCi/mL in the vehicle. Three randomly selected aliquots of each dose solution were analyzed for the determination of radiolabel concentration and homogeneity before and after dosing. Preparation of the doses were documented along with the nominal concentration in mg/mL, the specific activity, the number of mCi administered to each animal and the dose in mg/kg of body weight.

Each animal received 16 mg and 2 mCi via oral gavage. The dosing formulations were prepared one day prior to dosing and stored in a refrigerator set to maintain 2-10° C. prior to dose administration. The dose formulation was warmed to ambient temperature for dosing and maintained at ambient temperature with constant stirring during dose withdrawal.

Radioactivity Concentration/Homogeneity were determined in triplicate for concentration analysis and homogeneity testing from the dose formulation prior to and following dosing by liquid scintillation counting (LSC) of dose formulation aliquots. The Cynos used in these studies were from the stock colony, laboratory bred, and experimentally non-naïve. Nine males that were 2-6 years old and weighed >4 kg were used. Monkeys are commonly used in pharmacokinetic studies and are accepted by appropriate regulatory agencies. The total number of animals used in this study was the minimum required to properly achieve study objectives. This determination was based on established industry conventions for this type of study. Animals were received from their source and acclimated to the facility and husbandry conditions according to Facility SOPs. Prior to release to the study, a veterinary staff member reviewed the health status of the animals. Animals were housed individually in stainless steel cages during the time of dosing and sample collection, animal room and cage cleaning were performed according to testing facility SOPs, and Purina Certified Diet No. 5048 or equivalent was provided daily in amounts appropriate for the size and age of the animals. Tap water was available ad libitum, to animals in their home cages. The animals may be fasted as noted in subsequent section(s) of the protocol for dosing.

Animals that were considered healthy and acceptable for study were assigned to Study Groups using a computer-generated randomization procedure based on body weight to control bias. The animals were acclimated to the study room for a minimum of 3 days prior to initiation of dosing. Animals were acclimated to study-specific experimental procedures to identify and exclude from study those that exhibited increased levels of stress or resistance to the procedure, which were removed from the study group and replaced by a spare animal. Predose data (e.g. veterinary examinations, ophthalmology exams, etc.) were collected for selection of animals that were randomized to study, and the data was maintained in the study file. The monkeys were assigned to groups as shown in the table below and dosed orally. The day of dosing was considered as Day 1.

TABLE IV
Group assignments

| | | | Dose | | Termination |
|---|---|---|---|---|---|
| | | Number of | Level | Samples to be | Time Point |
| Group | Test Article | Monkeys | (mg) | Quantified | (hr) |
| 1 | [³H]PPS | 3 | 16 | Plasma, Urine | 96 |
| 2 | [³H]PPS-5 | 3 | 16 | Plasma, Urine | 96 |
| 3 | [³H]PPS-7 | 3 | 16 | Plasma, Urine | 96 |

Animals were fasted overnight prior to receiving a single oral dose of the test article. This frequency of administration is appropriate to determine the pharmacokinetics of the test articles. Doses were given by nasogastric gavage, a route that is consistent with the proposed route of administration in humans. Food was returned 3.5 to 4 hours postdose. The dose level was extrapolated from the human dose data for Elmiron® (active ingredient is PPS) and from a prior study of PPS that had been conducted in African Green monkeys using doses up to 400 mg. The volume of the dose was 8 mL/kg or less depending on body weight, which was based on the most recent body weight of each animal. The In-Live observations and measurements included observation for viability at least 4 hours apart throughout the study. Any animals observed to have pain or distress would have been brought to the attention of the Study Director and/or a staff veterinarian. Urine, feces, cage wash, and plasma were collected at the time points listed in the table below.

All samples collected during this study were placed in labeled containers. Each label contained the study number, animal number, sample type and collection interval, and date of collection. Plastic containers were used for sample storage. Cage wash samples were stored in a refrigerator set to maintain 2-10° C., pending analysis; following analysis, the samples were stored in a freezer set to maintain −20° C. Urine and plasma samples were stored in a freezer set to maintain −20° C. until analysis. Feces samples were stored in a freezer set to maintain −20° C. for possible future analysis.

Urine was collected continuously via a cage tray from each animal at predose and at 0-6, 6-12, 12-24, 24-48, 48-72, and 72-96 hours postdose. The cage tray was tilted to allow the urine to drain into the collection vessel. The weight of each urine sample was measured, and duplicate weighed aliquots of each urine sample were analyzed using LSC. Following LSC analysis, all remaining urine samples were stored in a freezer set at −20° C. for possible future analysis.

Urine samples were analyzed to determine the presence of volatile radioactivity by concentrating aliquots of the samples to dryness, quantitation of radioactivity by LSC, and then comparing the results with values obtained from the same samples that had not been concentrated.

Feces samples were collected continuously from each animal at predose and at 0-6, 6-12, 12-24, 24-48, 48-72, and 72-96 hours postdose. All fecal samples were stored in a freezer set at −20° C. until homogenized. The weight of each fecal sample was measured. HPLC water will be added to each fecal sample (approximately 2:1 water:feces ratio, v:w). After allowing the feces to absorb water, the feces will be homogenized by vortexing/shaking to form a slurry. The total homogenate weight will be obtained. The fecal homogenates will be stored in a freezer set at −20° C. for possible future analysis.

After each urine and fecal collection, a cage wash was performed. When animals were present, cages were rinsed with water. At termination, cages were rinsed with water, followed by a methanol rinse. The water and methanol rinses were combined for this analysis.

Cage wash samples were collected for radiation safety purposes, but were not analyzed for radiolabel content; these cage wash samples will be disposed of (if necessary) in compliance with all applicable laws regulating radiochemical materials.

Following blood collection, the samples were inverted several times and stored in a refrigerator set to maintain 2-10° C., pending centrifugation at set points of 5° C. for 10 minutes at 1500×g to obtain plasma. Plasma was then removed to another tube, and duplicate aliquots of each plasma sample were analyzed for radioactivity using LSC. Remaining plasma samples were stored in a freezer set to maintain −20° C. for possible future analysis.

Plasma samples were analyzed to determine the presence of volatile radioactivity by concentrating aliquots of the samples to dryness, quantitation of radioactivity by LSC, and then comparing the results with values obtained from the same samples that had not been concentrated.

The quantity of radiolabel found in the plasma was expressed as the nanogram equivalents of PPS, PPS-5, or PPS-7 per gram. Data from the analyses of the plasma radioactivity concentration data was used to calculate the following pharmacokinetic parameters for each test article. Noncompartmental analysis was conducted with WinNonlin, version 6.2, operating as a validated software system. Target time points were used if collections were made outside of the specified collection windows. Calculated parameters included: time (Tmax) to reach peak concentrations of radiolabel; the concentration (Cmax) of radiolabel at Tmax; total area under the concentration versus time curve (AUC) of areas of interest (i.e., AUC₀₋₄₈); and percent recovery of the applied radiolabeled dose (% AD) in the urine.

TABLE V
Sample collection time points

| | Collection Time |
|---|---|
| Sample | Points |
| Urine | Predose, 0-6, 6-12, 12-24, |
| | 24-48, 48-72, 72-96 |
| | hours |
| Feces | Predose, 0-6, 6-12, 12-24, |
| | 24-48, 48-72, 72-96 |
| | hours |
| Cage Wash | Predose, 0-6, 6-12, 12-24, |
| | 24-48, 48-72, 72-96 |
| | hours |
| Blood/plasma* | 0.5, 1, 2, 4, 6, 8, 10, |
| | 12, 24, 36, 48 hours |

*Blood samples (target of 2 mL) via venipuncture of the femoral (or other suitable) vein into tubes containing K₃EDTA anticoagulant.

All animals were euthanized at 96 hours postdose using an injection of ketamine followed by an injection of Fatal-Plus®.

The computer systems used on this study included, but were not be limited to, the following the WinNonlin version 6.2.1 validated system for pharmacokinetic calculations

Results

Previous BA/PK studies of PPS were limited by the lack of reliable PPS assays and the limited radioactivity doses permitted in humans. Thus, the use of 80-fold greater amounts per Kg of ³H-labeled PPS test items in these studies were designed to provide the most informative BA/PK data for PPS to date and the first such data for PPS fractions.

The BA/PK characteristics of the ³H-PPS test items established in monkeys were determined by first measuring total radioactivity in plasma and urine in triplicate for each test item.

Volatile radioactivity (˜90%) was removed from the total radioactivity results to analyze the radioactivity in the samples that was not tritiated water. In all measurements of original pre-administered test items, plasma samples, and urine samples approximately 90% of the radioactivity was volatile (tritiated water). The radioactivity in the samples was recalculated on the basis of the non-volatile specific radioactivity; the BA/PK characteristics of the non-volatile ³H-PPS test items established in Cynos are shown in Table VI.

TABLE VI

| Test | | Mean | | | Cumulative urinary |
|---|---|---|---|---|---|
| Items | Mean BA | Cmax ± | Mean Tmax ± | Mean AUC₍₀₋₄₈₎ ± | excretion_{(96 hr)} ± |
| (2mCi, | Relative | SD | SD | SD | SD |
| 16 mg) | to PPS* | (μg/mL) | (hr) | (μg * hr/mL) | (% of dose) |
| PPS | 1.0 | 0.835 ± 0.268 | 2.3 ± 1.2 | 9.734 ± 0.261 | 9.77 ± 1.28 |
| Fract 5 | 1.88 | 1.439 ± 0.136 | 27.3 ± 19.2 | 50.198 ± 4.137 | 18.36 ± 6.86 |
| Fract 7 | 2.06 | 1.726 ± 0.138 | 12.5 ± 20.4 | 54.899 ± 11.040 | 20.14 ± 14.37 |

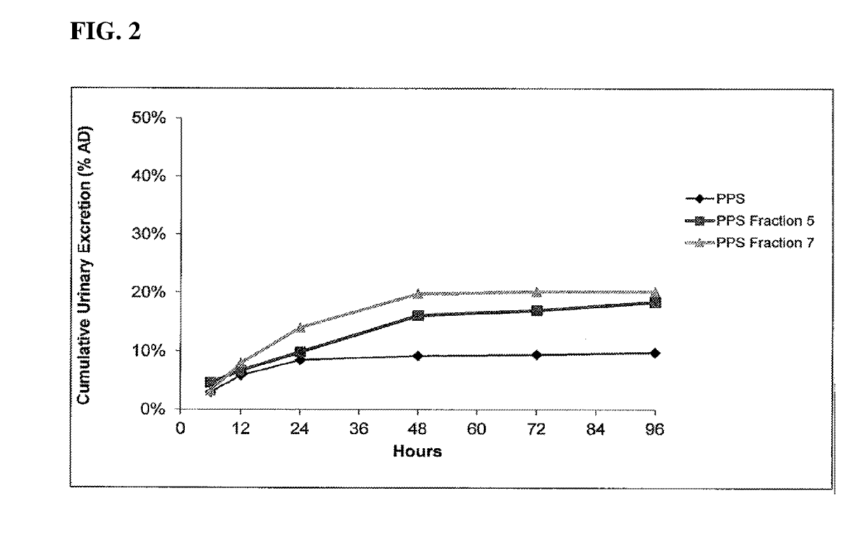

**Based on accumulated urinary excretion by 96 hr, as shown in FIG. 2.

Oral BA of Fractions 5 and 7 are similar, better than PPS, and persist longer in the circulation than PPS. We selected Fraction 5 as VTI-1968 because of its greater activity and oral BA.

Mean non-volatile cumulative urinary excretion radioactivity for PPS, Fraction 5, and Fraction 7 through 96 hr is shown in FIG. 2. The data points are urinary radioactivity from triplicate measurements for each test item from which the volatile radioactivity of tritiated water was removed as % of administered dose (% AD). As shown, the fractions have greater urinary excretion through 96 hours in comparison to unfractionated PPS.

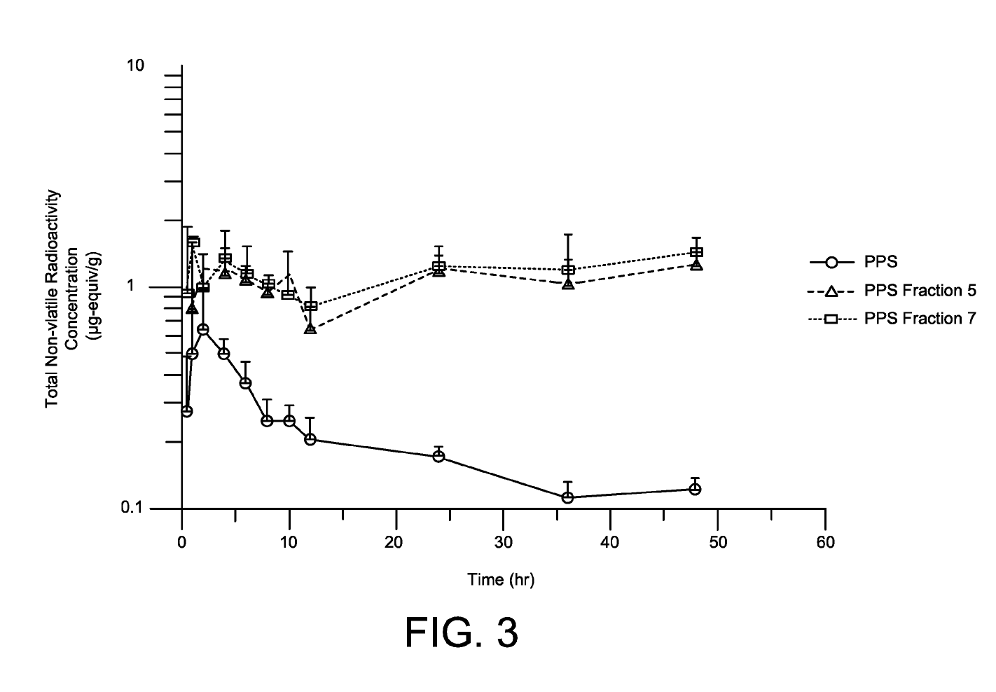

Mean non-volatile radioactivity concentrations for PPS, Fraction 5, and Fraction 7 are shown in FIG. 3. The data points are mean plasma radioactivity from triplicate measurements for each test item from which the volatile radioactivity of tritiated water was removed. As shown, the fractions have greater oral BA than PPS and remain bioavailable even after 48 hours in comparison to unfractionated PPS.

### Example 5: Selectin Blocking Specificity of PPS Fractions

This Example demonstrates that unfractionated PPS and specific PPS fractions are able to block molecular binding to P-selectin but not to L- or E-selectin. Properties of unfractionated PPS and Fractions 5 and 7, identified in the preceding Examples, were compared with respect to blocking activity against molecular adhesion of P-selectin. E-selectin, or L-selectin to sialyl Lewis A (sLeA).

Materials and Methods

The molecular P-, E-, and L-selectin blocking activities of unfractionated and fractionated PPS test items were determined using a modification of the assay for P-selectin binding to sialyl Lewis A (sLeA) (Kutlar, et al., (2012) Am J Hematol 87, 536-539 plus Online Supplemental Material). Concentrations of PPS test items ranging from 0.05 to 5000 μg/mL were mixed with P-selectin-Ig chimera, E-selectin-Ig chimera, or L-selectin-Ig chimera. After incubation and washing, selectin-Ig chimera bound to sLeX was quantified using goat anti-human Ig, color development, and a microplate reader. The percentage blocking by unfractionated PPS and each PPS fraction concentration and each selectin tested were graphed. Percent blocking was calculated as the mean OD of three wells (OD of sample−OD of blank)÷(OD of a 0 μg/ml PPS fraction−OD of blank).

This method for blocking of P-selectin and not or weakly blocking E-selectin or L-selectin is unique in its specificity for this polymeric carbohydrate class of agents.

Results

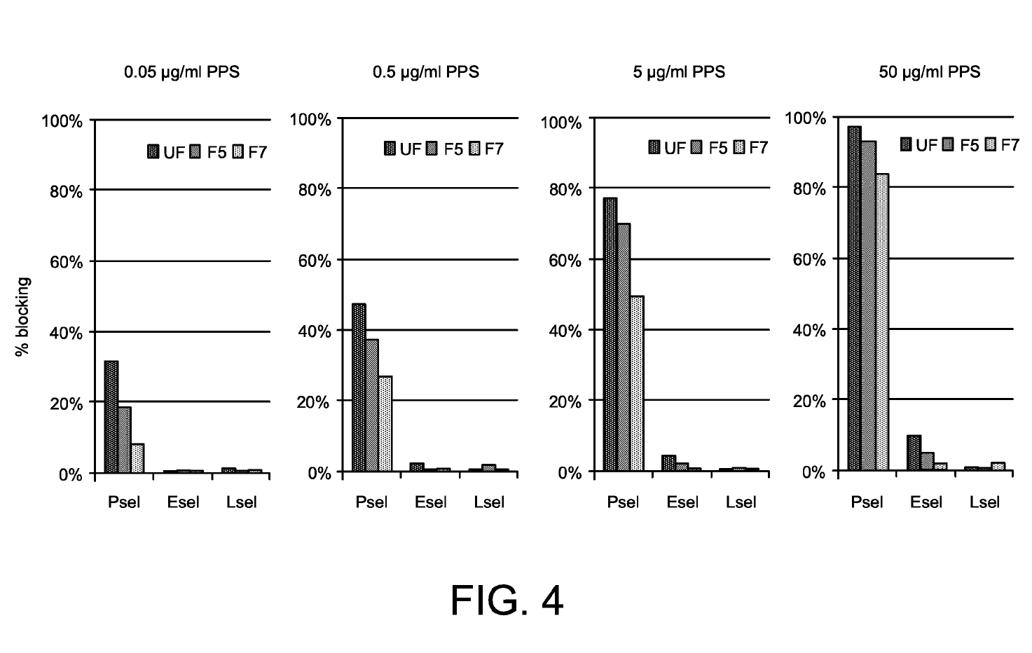

Results are shown in FIG. 4 and Table VII. Both unfractionated PPS as well as Fractions 5 and 7 are highly effective with respect to their ability to block P-selectin, but not L-selectin or E-selectin. As such, this demonstrates that unfractionated PPS and Fractions 5 and 7 are highly selective blockers of P-selectin. Furthermore, the concentration dependence of P-selectin blocking versus that of E- and L-selectin blocking indicates that, at concentrations anticipated from the expected oral doses, there would be virtually no E- or L-selectin blocking.

TABLE VII
Percent blocking by unfractionated PPS and by Fractions 5 and 7.

| μg/ml | Psel, UF | Psel, F5 | Psel, F7 | Esel, UF | Esel, F5 | Esel, F7 | Lsel, UF | Lsel, F5 | Lsel, F7 |
|---|---|---|---|---|---|---|---|---|---|
| 0.05 | 31.4% | 18.4% | 8.0% | −0.6% | 0.2% | −0.8% | 1.2% | −2.4% | 0.5% |
| 0.5 | 47.3% | 37.2% | 26.8% | 2.1% | −1.1% | 0.2% | −3.1% | 1.5% | −1.0% |
| 5 | 77.2% | 70.2% | 49.5% | 4.2% | 2.1% | −1.9% | −0.7% | 0.8% | −0.2% |
| 50 | 97.2% | 93.1% | 83.9% | 9.4% | 4.7% | 2.0% | 0.3% | −0.3% | 1.9% |

### Example 6: PPS Fractions can Improve P-Selectin Blocking Activity and Protect Against P-Selectin Induced Blood Flow Impairment in Mice

This Example demonstrates that specific PPS fractions can improve P-selectin blocking activity compared to unfractionated PPS and protect against P-selectin induced blood flow impairment in vivo. Properties of PPS and Fraction 5, identified in the preceding Examples, are compared with respect to blocking P-selectin as measured by effect on baseline blood flow and by protective effects against P-selectin induced stoppage of blood flow using computer-assisted intravital microscopy (CAIM) in a sickle cell chimeric mouse system.

Materials and Methods

The P-selectin blocking activity as measured by effect on baseline blood flow and protective effects against blood flow stoppage of PPS and Fraction 5 is determined using CAIM in the sickle cell chimeric mouse system, as we have described. The system uses CAIM to determine the flow velocity of XRITC-labeled sickle red blood cells (SRBC) from Paszty sickle cell mice (Paszty, et al 1997) that are suspended in phosphate-buffered saline to a hematocrit of 25% and 50 μL infused by tail vein injection into recipient mice to obtain a 1:200 in vivo ratio of labeled to unlabeled RBC and minimize hemodynamic changes from the infused volumes. Recipient mice are C57BL/6, Paszty sickle cell, or P-selectin null (Bullard, et al 1995) mice that are anesthetized with intraperitoneal sodium pentobarbital or isoflurane inhalation. The velocity of flow is determined by CAIM imaging flow in mucosal-intestinal small venules of externalized abdominal viscera using in-house developed software (Cheung, et al 2002).

First, the velocity of baseline blood flow in untreated Paszty sickle cell mice (n=3), C57BL/6 mice (n=3), P-selectin null mice (n=3) and each of these mice pretreated with PPS (n=9) or Fraction 5 (n=9), respectively, is determined. Amounts of PPS and Fraction 5 ranging from 10 μg to 1 μg are dissolved in 0.2 mL PBS and administered by gastric gavage to host mice 30-60 minutes before velocity measurements are made.

Secondly, the time to stoppage of flow after activation of endothelial cells to express P-selectin by suffusion of the mesentery with PAR-1 agonist peptide (TFLLR-NH2) is measured. The protective effects of pretreating recipient C57BL/6 host mice with PPS or Fraction 5 before PAR-1 induced blood flow stoppage is determined in triplicate and the time to flow stoppage and is compared to results in recipient C57BL/6 mice that were not pretreated. Amounts of PPS and Fraction 5 ranging from 10 μg to 1 μg are dissolved in 0.2 mL PBS and administered by gastric gavage to host mice 30-60 minutes before PAR-1 suffusion onto the mesentery. P-selectin knockout mice recipients are used as negative controls.

Results

Table VIII shows predicted baseline flow velocities in medium sized (20-40 μm diameter) venules of untreated C57BL/6 recipient mice, C57BL/6 recipient mice pretreated with PPS or Fraction 5, untreated P-selectin null recipient mice, and P-selectin null recipient mice pretreated with PPS or Fraction 5 after injection of XRITC-labeled sickle red blood cells.

TABLE VIII
Predicted Velocities of Blood Flow According to Endothelial P-selectin
Activity

| | Velocity of RBC |
|---|---|
| | flow |
| | Mean ± SEM |
| Study Groups | (mm/sec.) |
| Untreated C57BL/6 recipients | 3.0 ± 0.2 |
| C57BL/6 recipients pretreated with PPS | 3.0 ± 0.2 |
| C57BL/6 recipients pretreated with Fraction 5 | 3.0 ± 0.2 |
| Untreated P-selectin null mice recipients | 3.0 ± 0.2 |
| P-selectin null mice pretreated with PPS | 3.0 ± 0.2 |
| P-selectin null pretreated with Fraction 5 | 3.0 ± 0.2 |
| Untreated sickle cell mouse recipients | 1.5 ± 0.3 |
| Sickle cell mouse recipients pretreated with PPS | 3.0 ± 0.2 |
| Sickle cell mouse recipients pretreated with Fraction 5 | 3.0 ± 0.2 |

Baseline blood flow velocity in untreated sickle cell mouse recipients is predicted to be lower than in recipient mice in which endothelial cell P-selectin is not activated (sickle cell or C57BL/6 mouse recipients), absent (P-selectin null mouse recipients), or any recipients in which endothelial P-selectin has been blocked by PPS or Fraction 5. P-selectin blocking with PPS or Fraction 5 is predicted to have an effect on baseline blood flow velocity equal to the absence of P-selectin in P-selectin null recipient mice.

Table IX shows predicted times to stoppage of flow after activation of mucosal intestinal venules by suffusion of the mesentery with PAR-1 agonist peptide measured in medium sized (20-40 μm diameter) venules of untreated C57BL/6 recipient mice, C57BL/6 recipient mice pretreated with PPS or Fraction 5, untreated P-selectin null recipient mice, and P-selectin null recipient mice pretreated with PPS or Fraction 5.

TABLE IX
Predicted Protective Effects of PPS and Fraction 5 Against Blood Flow Stoppage
Mean ± SEM (min.)

| | | | | P-selectin | |
|---|---|---|---|---|---|
| | C57BL/6 | C57BL/6 | Untreated P- | null | P-selectin null |
| Untreated | pretreated | pretreated | selectin null | pretreated | pretreated with |
| C57BL/6 | with PPS | with Fraction 5 | mice | with PPS | Fraction 5 |
| 3.5 ± 1.0 | No stoppage | No stoppage | No stoppage | No stoppage | No stoppage at 8 |
| | at 8 | at 8 | at 8 | at 8 | |

Time to stoppage of flow in untreated C57BL/6 mouse recipients is shorter than in C57BL/6 recipient mice in which endothelial P-selectin is absent (P-selectin null mouse recipients) or is blocked by PPS or Fraction 5. P-selectin blocking with PPS or Fraction 5 has an effect on preventing stoppage of blood flow equal to the absence of P-selectin in P-selectin null recipient mice.

### REFERENCES

Bullard, D. C., Qin, L., Lorenzo, I., Quinlin, W. M., Doyle, N. A., Bosse, R., Vestweber, D., Doerschuk, C. M. & Beaudet, A. L. (1995) P-selectin/ICAM-1 double mutant mice: acute emigration of neutrophils into the peritoneum is completely absent but is normal into pulmonary alveoli. J Clin Invest, 95, 1782-1788.
Cheung, A. T., Chen, P. C., Larkin, E. C., Duong, P. L., Ramanujam, S., Tablin, F. & Wun, T. (2002) Microvascular abnormalities in sickle cell disease: a computer-assisted intravital microscopy study. Blood, 99, 3999-4005.
Embury, S. H., Matsui, N. M., Ramanujam, S., Mayadas, T. N., Noguchi, C. T., Diwan, B. A., Mohandas, N. & Cheung, A. T. (2004) The contribution of endothelial cell P-selectin to the microvascular flow of mouse sickle erythrocytes in vivo. Blood, 104, 3378-3385.
Paszty, C., Brion, C. M., Manci, E., Witowska, H. E., Stevens, M. E., Mohandas, N. & Rubin, E. M. (1997) Transgenic knockout mice with exclusively human sickle hemoglobin and sickle cell disease. Science, 278, 876-878.

### Example 7: PPS Fractions Provide P-Selectin Blocking Activity and Improve Abnormal Blood Flow of Asymptomatic Patients with Sickle Cell Disease

This Example demonstrates that specific PPS fractions are able to provide P-selectin blocking activity and improve the abnormal blood flow of asymptomatic patients with sickle cell disease. The effects of PPS and Fraction 5 identified in the preceding Examples on blood flow velocity in small vessels are tested using computer-assisted intravital microscopy (CAIM) of conjunctival venules and Laser Doppler velocimetry (LDV) to characterize post-occlusive reactive hyperemia (PORH) in small subcutaneous forearm vessels. The abnormal baseline blood flow in patients with sickle cell disease is reflected by lower than normal blood flow velocity with CAIM and a longer than normal duration of PORH with LDV. Effective P-selectin blocking by PPS or Fraction 5 is predicted to return these abnormal measures toward normal.

Materials and Methods

The effect of PPS and Fraction 5 on blood flow velocity in small vessels is determined using CAIM to assess blood flow velocity in conjunctival venules and LDV to assess PORH in small subcutaneous vessels of the forearm, as we and others have described (Bachir, et al 1993, Cheung, et al 2002, Kutlar, et al 2012, Rodgers, et al 1990). The CAIM system measures blood flow velocity noninvasively in selected conjunctival small venules directly in real time. The LDV system determines changes in blood flow noninvasively by characterizing the duration of hyperemic responses after periods of blood flow occlusion, the most informative parameter of which is the time from stoppage of occlusion to half the peak flow (TH1). The CAIM and LDV methods are used simultaneously to compare blood flow in asymptomatic patients with sickle cell disease before and after oral administration of PPS or Fraction 5 test items.

Baseline pretreatment blood flow in asymptomatic patients with sickle cell disease (n=6) and healthy nonsickle controls (n=3) is determined using CAIM and LDV simultaneously. Digital recordings of blood flow velocity in selected conjunctival small venules are made using CAIM, and the recordings are labeled for later blinded interpretation. LDV recordings are made using three thermal probes set at 33° C. affixed to the forearm for one minute after the flow is stable, during three minutes of flow occlusion by inflating a blood pressure cuff on the upper arm to 200 mm Hg, throughout the period of hyperemia, and until the flow returns to baseline, and the recordings are labeled and saved for blind comparisons of time to half the peak flow. A battery of flow parameters is assessed by the software system of the LDV unit, among these the TH1 is the most informative about changes in blood flow, and this example uses that measurement to demonstrate the expected findings.

Escalating oral 100, 200, and 300 mg doses of PPS and 22, 44, and 66 mg doses of Fraction 5 are administered to each subject after baseline blood flow measurements are made with one-week washout periods between each dose. Subsequent blood flow measurements are made at 0.5, 1, 2, 4, 6, 8, 12, 24, 48 and 72 hours after administration of PPS or Fraction 5.

On each study day a test subject is administered PPS in early morning, according to the escalating dose schedule above, beginning with the lowest dose. After a one-week washout period that individual returns for the next higher dose, and after another week that individual receives the top dose of PPS. One week later the test subject begins with the lowest dose of Fraction 5 and continues weekly as with PPS. After 12-hour measurements, the test subject is discharged home and returns the next morning for 24-hour measurement, the next morning for 48-hour measurements, and the next morning for 72-hour measurements, which are the last.

Results

The mean data collected according to method of measurement, test item, dose, and dose schedule is shown below in Tables X and XI.

TABLE X
Predicted Blood Flow Velocities Measured Using CAIM Mean ± SEM (mm/sec.)

| | | PPS | PPS | PPS | Fraction 5 | Fraction 5 | Fraction 5 |
|---|---|---|---|---|---|---|---|
| Schedule | | 100 mg | 200 mg | 300 mg | 22 mg | 44 mg | 66 mg |
| Pretreatment | | 1.5 ± 0.8 | 1.5 ± 0.8 | 1.5 ± 0.8 | 1.5 ± 0.8 | 1.5 ± 0.8 | 1.5 ± 0.6 |
| 0.5 | hr | 1.5 ± 0.8 | 1.5 ± 0.8 | 1.5 ± 0.8 | 1.8 ± 0.8 | 2.0 ± 0.6 | 2.2 ± 0.6 |
| 1 | hr | 1.5 ± 0.8 | 1.5 ± 0.8 | 2.0 ± 0.6 | 2.0 ± 0.6 | 2.5 ± 0.6 | 2.5 ± 0.6 |
| 2 | hr | 1.8 ± 0.8 | 1.8 ± 0.8 | 2.5 ± 0.6 | 2.5 ± 0.6 | 2.5 ± 0.6 | 2.5 ± 0.6 |
| 4 | hr | 1.5 ± 0.8 | 1.8 ± 0.8 | 2.5 ± 0.6 | 2.5 ± 0.6 | 2.5 ± 0.6 | 2.5 ± 0.6 |
| 6 | hr | 1.5 ± 0.8 | 1.5 ± 0.8 | 2.5 ± 0.6 | 2.5 ± 0.6 | 2.5 ± 0.6 | 2.5 ± 0.6 |
| 8 | hr | 1.5 ± 0.8 | 1.5 ± 0.8 | 2.2 ± 0.6 | 2.2 ± 0.6 | 2.5 ± 0.6 | 2.5 ± 0.6 |
| 12 | hr | 1.5 ± 0.8 | 1.5 ± 0.8 | 1.8 ± 0.6 | 2.0 ± 0.6 | 2.2 ± 0.6 | 2.5 ± 0.6 |
| 24 | hr | 1.5 ± 0.8 | 1.5 ± 0.8 | 1.5 ± 0.8 | 1.5 ± 0.6 | 1.8 ± 0.6 | 2.5 ± 0.6 |
| 48 | hr | 1.5 ± 0.8 | 1.5 ± 0.8 | 1.5 ± 0.8 | 1.5 ± 0.8 | 1.5 ± 0.6 | 1.8 ± 0.6 |
| 72 | hr | 1.5 ± 0.8 | 1.5 ± 0.8 | 1.5 ± 0.8 | 1.5 ± 0.8 | 1.5 ± 0.8 | 1.5 ± 0.8 |

The lowest dose of PPS induces no therapeutic response. The middle dose of PPS induces a slight and prolonged response. The top dose of PPS normalizes blood flow beginning at 1 hour after dosing and lasting 8 hours. The lowest dose of Fraction 5 induces a response similar to the top dose of PPS except that it begins sooner and lasts longer. The middle dose of Fraction 5 induces a stronger early response that lasts longer than the lowest dose. The top dose of Fraction 5 normalizes blood flow beginning at 0.5 hour, and this response persists for 24 hours. The healthy non-sickle control subjects have normal flow measurements before and during treatment that range from 2.4-2.6 mm/sec.

TABLE XI
Predicted Duration of PORH Measured Using LDV TH1 (sec.)

| | | PPS | PPS | PPS | Fraction 5 | Fraction 5 | Fraction 5 |
|---|---|---|---|---|---|---|---|
| Schedule | | 100 mg | 200 mg | 300 mg | 22 mg | 44 mg | 66 mg |
| Pretreatment | | 8 ± 3 | 8 ± 3 | 8 ± 3 | 8 ± 3 | 8 ± 3 | 8 ± 3 |
| 0.5 | hr | 8 ± 3 | 8 ± 3 | 8 ± 3 | 6 ± 3 | 5 ± 3 | 4 ± 3 |
| 1 | hr | 8 ± 3 | 8 ± 3 | 5 ± 2 | 4 ± 3 | 3 ± 2 | 3 ± 2 |
| 2 | hr | 8 ± 3 | 5 ± 3 | 3 ± 2 | 3 ± 3 | 3 ± 2 | 3 ± 2 |
| 4 | hr | 8 ± 3 | 5 ± 3 | 3 ± 2 | 3 ± 3 | 3 ± 2 | 3 ± 2 |
| 6 | hr | 8 ± 3 | 8 ± 3 | 3 ± 2 | 3 ± 3 | 3 ± 2 | 3 ± 2 |
| 8 | hr | 8 ± 3 | 8 ± 3 | 4 ± 2 | 4 ± 3 | 3 ± 2 | 3 ± 2 |
| 12 | hr | 8 ± 3 | 8 ± 3 | 6 ± 2 | 5 ± 3 | 4 ± 2 | 3 ± 2 |
| 24 | hr | 8 ± 3 | 8 ± 3 | 8 ± 3 | 7 ± 3 | 5 ± 3 | 3 ± 3 |
| 48 | hr | 8 ± 3 | 8 ± 3 | 8 ± 3 | 8 ± 3 | 8 ± 3 | 7 ± 3 |
| 72 | hr | 8 ± 3 | 8 ± 3 | 8 ± 3 | 8 ± 3 | 8 ± 3 | 8 ± 3 |

The lowest dose of PPS induces no therapeutic response. The middle dose of PPS induces a modest and prolonged response. The top dose of PPS normalizes blood flow beginning at 1 hour after dosing and lasting 8 hours. The lowest dose of Fraction 5 induces a response similar to the top dose of PPS except that it begins sooner and lasts longer. The middle dose of Fraction 5 induces a stronger early response that lasts longer than the lowest dose. The top dose of Fraction 5 normalizes blood flow beginning at 0.5 hour, and this response persists for 24 hours. The healthy non-sickle control subjects have normal flow measurements before and during treatment that range from 2.4-2.6 mm/sec.

Both test items improve sickle cell blood flow in small vessels. Fraction 5 has greater activity than PPS, and its effect begins sooner and persists longer.

### REFERENCES

Bachir, D., Maurel, A., Beuzard, Y., Razavian, M., Kraiem, A., Portos, J. L. & Galacteros, F. (1993) Improvement of microcirculation abnormalities in sickle cell patients upon buflomedil treatment. Microvasc Res, 46, 359-373.
Cheung, A. T., Chen, P. C., Larkin, E. C., Duong, P. L., Ramanujam, S., Tablin, F. & Wun, T. (2002) Microvascular abnormalities in sickle cell disease: a computer-assisted intravital microscopy study. Blood, 99, 3999-4005.
Kutlar, A., Ataga, K. I., McMahon, L., Howard, J., Galacteros, F., Hagar, W., Vichinsky, E.,
Cheung, A. T., Matsui, N. & Embury, S. H. (2012) A potent oral P-selectin blocking agent improves microcirculatory blood flow and a marker of endothelial cell injury in patients with sickle cell disease. Am J Hematol, 87, 536-539 plus Online Supplemental Material.
Rodgers, G. P., Schechter, A. N., Noguchi, C. T., Klein, H. G., Nienhuis, A. W. & Bonner, R. F. (1990) Microcirculatory adaptations in sickle cell anemia: reactive hyperemia response. Am J Physiol, 258, H113-120.

### Example 8: Bioanalytical Method for Quantifying PPS Fractions in Blood

To support future pre-clinical and clinical studies, a bioanalytical method is needed to quantify PPS in rat, monkey (Cynomolgus), and human blood. The purpose of this Example is to test monoclonal antibodies (mAbs) and ELISAs for detecting and quantifying PPS and Fraction 5 and, if necessary, to modify the protocol to increase sensitivity and/or reduce background detection.

### I. Initial Development of ELISA Assay

Materials and Methods

A sandwich ELISA for detecting PPS was developed by BioRad (Hercules, Calif.) using HuCal® phage display antibody generation and screening. The mAbs were generated using unfractionated PPS as the antigen. The original protocol called for the following steps:
1. Coat. Each well was coated with capture Ab at 20 μL/well. 5 μg/mL Bio-Rad24722.1 in PBS was coated in each well of a 384-well Nunc Maxisorp MTP black flat bottom PS plate (ThermoScientific, 103959910). Incubate overnight, 4° C.
2. Wash. Wash twice with wash buffer (PBS w/0.05% Tween 20; PBST)
3. Block. 100 μL/well of block buffer (5% BSA in PBST). Incubate 1 hr, RT.
4. Wash. Wash twice with wash buffer.
5. Add Antigen. Add 20 μL/well of antigen in matrix (either PBST or 10% plasma in PBST). Incubate 1 hr, RT.
6. Wash. Wash 5× with wash buffer.
7. Detect Ab. Add 20 μL/well of 2 μg/mL Bio-Rad24711.1-HRP in PBST. Incubate 1 hr, RT.
8. Wash. Wash 10× with wash buffer.
9. Detection. Add 20 μL/well of QuantaBlu (ThermoScientific 15169) substrate (use as per QuantaBlu instructions). Read at excitation 320±25 nm, emission at 430±35 nm.

Results:

This method gave a Lower Limit of Detection (LLOD) (2× signal/background) of 0.5 ng/mL PPS in buffer, 0.5 ng/mL in 10% rat plasma in buffer, 2 ng/mL in 10% cynomolgus plasma in buffer, and 4 ng/mL in 10% human plasma in buffer.

Detection of Fraction 5 was less sensitive with this method and these reagents. The LLOD in buffer, 10% human plasma, 10% rat plasma, and 10% cynomolgus plasma was 1 ng/mL, 8 ng/mL, 2 ng/mL, and 8 ng/mL, respectively. This represents approximately half the sensitivity of unfractionated PPS. As the heterogeneity of unfractionated PPS is primarily by chain length, it is unlikely that epitope specificity is the cause of the difference in detection.

Nevertheless, this method was substantially improved over previous methods to detect and quantify PPS in blood such as polyclonal Ab-based ELISA methods for which sensitivity in plasma was lost below 200 ng/mL.

### II. Determination of the Optimal Antigen Matrix for ELISA Detection

This study determined whether higher proportions of plasma could be used in the matrix and whether plasma or serum was the better matrix for the PPS ELISA assay. In the development of polyclonal ELISAs, it was determined that plasma concentrations higher than 10% caused substantially less sensitivity in the assay. Without being bound by theory, it is possible that soluble P selectin might be present at higher levels in serum than in plasma. As such, these experiments determine if sensitivity of the assay is improved using either serum or plasma.

Materials and Methods

The ELISA method discussed above was used for this experiment, with modifications. PPS was mixed with human or rat sera or plasma for 0, 1, or 2 days at 5° C. prior to the ELISA experiments. In some experiments, the times of incubation varied from that described above (steps 1, 3, 5, and 7 of the method described in Section I) from 30, 45, 60, 90, 120 minutes, or overnight. Finally, in some experiments, the concentration of serum was 10%, 20%, or 40%.

Results:

In two experiments, the times of incubation for steps within the ELISA (steps 1, 3, 5, and 7 of the method described in Section I.B) were assessed. The following incubations were determined to be optimal with regards to the sensitivity of the assay:
1. Capture Ab: overnight, 4° C.
3. Block: 90 min, RT.
5. Antigen: 120 min, RT.
7. Detection Ab: 60 min, RT.

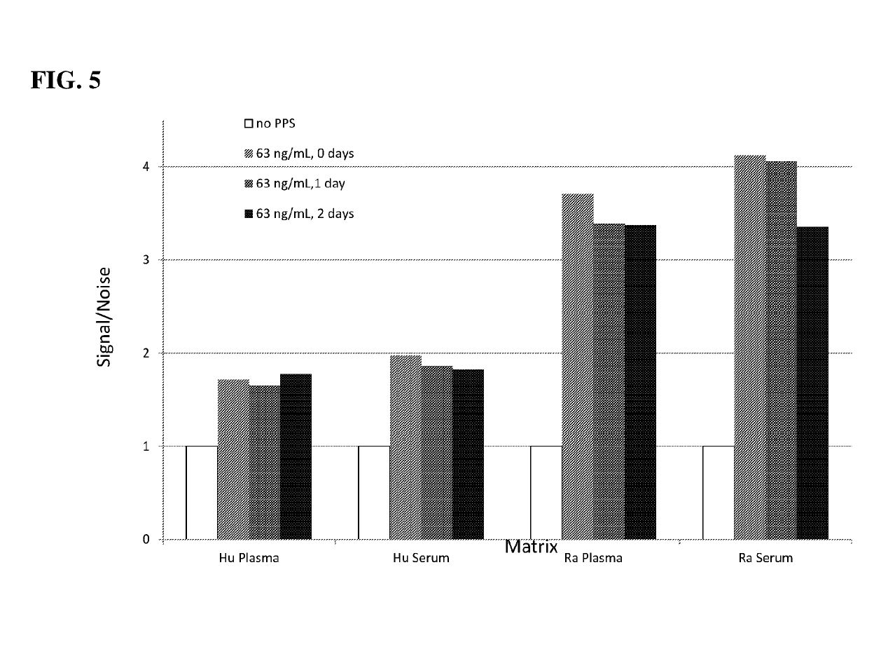

Two experiments were performed to determine the effect of matrix including the matrix as plasma vs. serum and the incubation of PPS with matrix. PPS in Rat matrices were detected more easily than PPS in human matrices. There was a slight trend of better PPS detection in serum as compared to plasma (FIG. 5). This was not statistically significant and was very slight, but was true in all experiments. PPS was detected better in all matrices when used right after mixing than after 1 or 2 days of incubation.

In two experiments, different concentrations of human and rat serum were used. Concentrations higher than 10% substantially impaired PPS detection for both human and rat serum.

Although the difference in detection sensitivity between plasma and serum was slight and not statistically significant, it was consistent. Thus for all subsequent experiments, serum rather than plasma was used. The effect of reduced PPS detection in matrices that have been incubated 1 or 2 days could be explained by increased interaction of PPS to substances within the matrix. Samples drawn from patients or animals given PPS in clinical or pre-clinical studies will have time for PPS to interact with interfering substances in blood. To generate accurate standard curves for such studies, it is recommended that PPS-matrix mixtures be incubated at 5° C. overnight.

### III. Determination of the Effect of Protease on the Potential Blood Interfering Factor(s)

This study determined whether protease treatment of the antigen in plasma or serum matrix would provide an increase in the sensitivity of the assay.

Materials and Methods

The protocol outlined in section I above was used with modifications. PPS was mixed with human or rat sera and treated with Pronase (500 U per 100 μl serum) for 1, 24, or 48 hours at room temperature or 37° C. Subsequently, Protease Inhibitor Cocktail III (EMD Millipore Biosciences 539134) was used to stop the proteolysis prior to being used as antigen in the ELISA.

Results:

Initial experiments for the effect of proteolysis included multiple incubation time and temperature experiments using only PPS as the antigen. Optimal incubation time and temperature was determined in these experiments to be overnight (18-20 hours) at 37° C.

After incubation with protease, a thin, white precipitate developed. Thus, the sample applied to the assay was a suspension rather than a solution. The precipitate was removed during the wash steps following antigen incubation.

In the final experiment of this study, the method, including protease treatment was tested for 5 concentrations of PPS and Fraction 5:0, 2, 6.3, 20, and 63 ng/mL. The incubation times and temperatures used in this experiment were the optimal settings found in the matrix study (see above, Section II) and in the first part of this study.

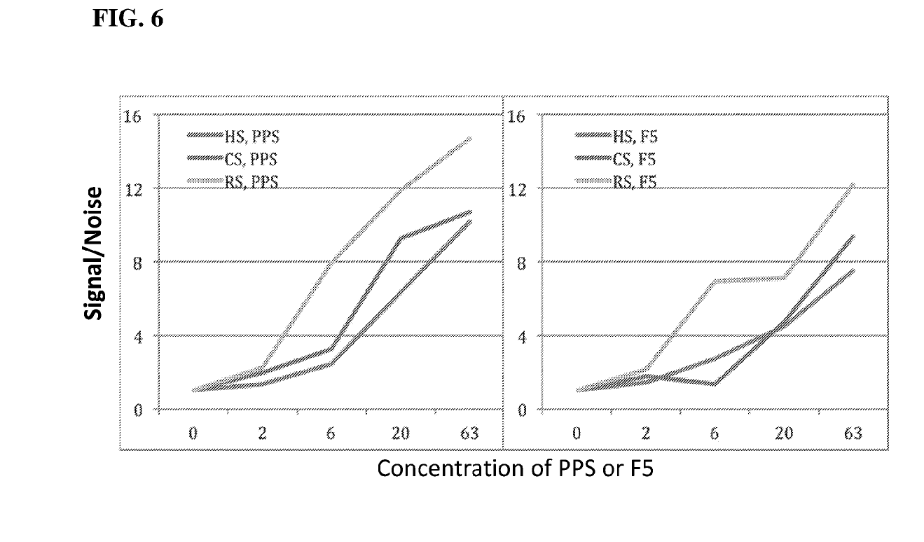

The plots of PPS and Fraction 5 in rat serum (“RS, PPS” and “RS, F5”, respectively) were similar to those described in Section I. In general, protease treatment provided more sensitivity for PPS and Fraction 5 in human and Cynomolgus sera, compared to the sensitivity seen in Section I (FIG. 6). The baseline (no antigen) levels of fluorescence for human, rat, and cynomolgus monkeys were 6818157, 3597614, and 4036989, respectively. As noted above, these values are higher than samples that are not protease treated, but they are less dissimilar than reported using the protocol in section I, which they were 553, 199, and 206.

Protease treatment increased the sensitivity of the PPS ELISA. Without being bound to theory, it is possible that the protease treatment makes the sera of the three species more similar by destroying proteins that may be more prevalent in one or the other. Because the matrices, human and cynomolgus sera that provide less sensitivity to ELISA are affected more by protease treatment than rat serum, this would indicate that proteins in those sera are a major source of interference in the assay.

IV. Determination of the Effect of Chondroitinase on the Potential Blood Interfering Factor(s)

This study determined whether chondroitinase (CS) treatment of the antigen in plasma or serum matrix would provide an increase in the sensitivity of the assay. Bioanalytical methods for detecting heparinoids in plasma found that chondroitin sulfate (CS) interfered with the assay (Mousa, et al, 2007, J Clin Pharmacol 47: 1508-1520). Cross-reactivity tests demonstrated that the two antibodies selected for the sandwich ELISA did not detect CS. Therefore, it was further assessed whether chondroitinase treatment of the matrix improved detection of PPS.

Materials and Methods:

The protocol outlined in section I above was used with modifications. PPS was mixed with human or rat sera and treated with Pronase (50 U per 100 μl serum) for 1, 24, or 48 hours at room temperature or 37° C., after which Protease Inhibitor cocktail was used to stop the proteolysis prior to being used as antigen in the ELISA.

Results:

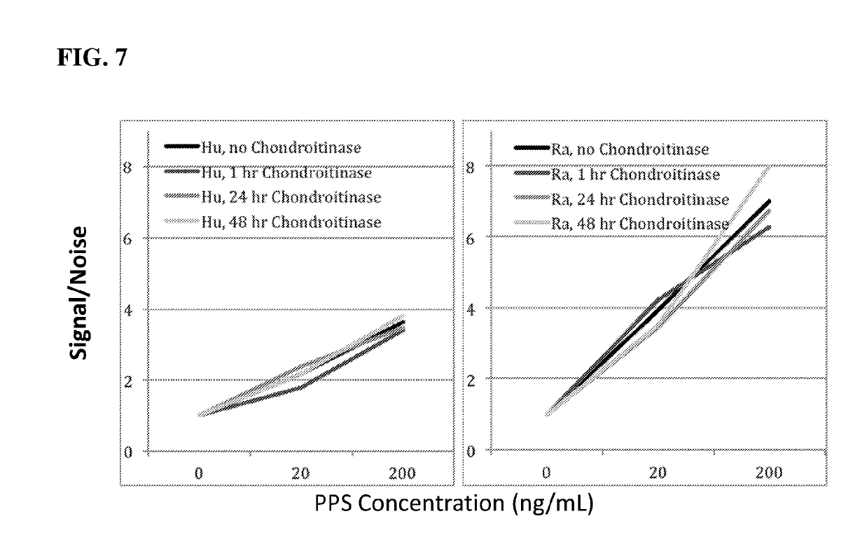

Two experiments were performed for this study, both of which had similar results. The data presented here are from the second assay. As shown in FIG. 7, sera with PPS treated with chondroitinase were similar to that without treatment for both human and rat sera. There was no effect on background as the fluorescence of wells with 0 PPS samples were similar for the untreated and treated samples.

Chondroitinase treatment of PPS in serum caused no appreciable change in signal in the ELISA method. The likely explanation for this is that chondroitinase-sensitive components have no effect on the ELISA detection of PPS. This is consistent with chondroitin sulfate having been one of the negative selection antigens used for the generation of the mAbs and the absence of cross-reactivity when Bio-Rad assessed antibody affinities. There is no indication for further study of chondroitinase.

### V. Determination of the Effect of Chloroform/Ethanol Extraction

These experiments determined whether chloroform/ethanol extraction of the antigen in plasma or serum matrix would provide an increase in the sensitivity of the assay. This method is based on one by Mielke, et al. to purify heparin from human plasma (Mielke, et al., 1999, Clin Appl Thrombosis/Hemostasis, 5(4): 267-76, the disclosure of which is incorporated by reference herein).

Materials and Methods:

The protocol outlined in section I above was used with modifications. PPS in serum was mixed with chloroform and then precipitated with ammonium acetate and ethanol. The mixture was centrifuged and the pellet was dehydrated then redissolved in PBS.

Results:

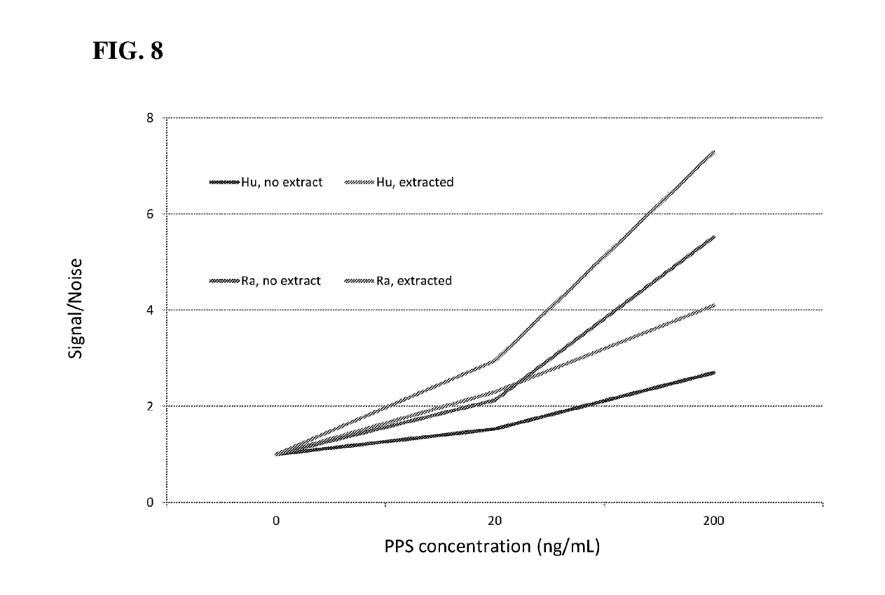

Two experiments were performed for this study, each of which had similar results. The data presented here are from the second assay. As shown in FIG. 8, extraction increased the sensitivity of the assay for PPS in both human and rat sera. There was no effect on background as the fluorescence of wells with 0 PPS samples were similar for the extracted and non-extracted samples.

The precipitate did not completely dissolve after dehydration and redissolution. The sample used for this study was a thin suspension. The suspended material was removed during the wash steps following antigen incubation.

Chloroform/ammonium acetate/ethanol extraction of PPS from serum causes an increase in signal in the ELISA method. In the research of Mielke, et al, this method was described as a way to purify heparin from plasma for physical chemical analysis. It is possible that this method is another way to separate PPS from protein. If that is the case, it causes less background fluorescence issues than the proteolysis method. Like the proteolysis method, the extraction adds a day to the assay.

### VI. Optimized Detection Method

Taking all of the above into consideration, the derived optimized assay is as follows:
1 Protease treatment. In biohazard cabinet, pipet 70 μL of Ag in serum into a sterile 1.6 mL microcentrifuge tube, add 10 μL of 50 kU/mL Pronase in PBS (EMD Millipore Biosciences 537088-5 KU). Vortex, then incubate at 37° C., overnight (at least 10 hours).
2. Stop protease. Add 20 μL Protease Inhibitor Cocktail III (EMD Millipore Biosciences 539134) to each tube. Vortex, then incubate 1 hr, RT.
3. Chloroform extraction. Add 100 μL chloroform to each tube. Vortex to mix.
4. Precipitation. Add 50 μL of 10 M Ammonium Acetate to each tube. Vortex to mix. Add 750 μL of 95% ethanol. Vortex, then incubate on ice for 30 min.
5. Isolation of the pellet. Centrifuge at 14 kG for 10 min at 5° C. Discard the supernatant. Dry pellet in a centrifugal evaporator overnight.
6. Capture Ab. 20 UL/well of 5 μg/mL Bio-Rad24722.1 in PBS was coated in each well of a 384-well Nunc Maxisorp MTP black flat bottom PS plate (ThermoScientific, 103959910). Incubate overnight, 4° C.
7. Wash. Wash plate twice with wash buffer (PBS w/0.05% Tween 20; PBST).
8. Block. 100 UL/well of block buffer (5% BSA in PBST). Incubate 90 min, RT.
9. Reconstitution of Ag. Dissolve each Ag pellet in 70 μL PBS-Tween.
10. Wash. Wash twice with wash buffer.
11. Antigen. 20 UL/well of antigen in matrix (either PBST or 10% plasma in PBST). Incubate 2 hr, RT.
12. Wash. Wash 5× with wash buffer.
13. Detection Ab. 20 μL/well of 2 μg/mL Bio-Rad24711.1-HRP in PBST. Incubate 1 hr, RT.
14. Wash. Wash 10× with wash buffer.
15. Substrate. 20 μL/well of QuantaBlu (ThermoScientific 15169) substrate (use as per QuantaBlu instructions). Read at excitation 320±25 nm, emission at 430±35 nm.

### REFERENCES

1. He, X. Y., Xu, Z., Melrose, J., Mullowney, A., Vasquez, M., Queen, C., Vexler, V., Klingbeil, C., Co, M. S., and Berg, E. L. (1998) Humanization and pharmacokinetics of a monoclonal antibody with specificity for both E- and P-selectin, J. Immunol. 160, 1029-1035.
2. Tardif, J. C., Tanguay, J. F., Wright, S. S., Duchatelle, V., Petroni, T., Gregoire, J. C., Ibrahim, R., Heinonen, T. M., Robb, S., Bertrand, O. F., Cournoyer, D., Johnson, D., Mann, J., Guertin, M. C., and L'Allier, P. L. (2013) Effects of the P-selectin antagonist inclacumab on myocardial damage after percutaneous coronary intervention for non-ST-segment elevation myocardial infarction: results of the SELECT-ACS trial, J Am Coil Cardiol 61, 2048-2055.
3. Gutsaeva, D. R., Parkerson, J. B., Yerigenahally, S. D., Kurz, J. C., Schaub, R. G., Ikuta, T., and Head, C. A. (2011) Inhibition of cell adhesion by anti-P-selectin aptamer: a new potential therapeutic agent for sickle cell disease, Blood 117, 727-735.
4. Imai, Y., True, D. D., Singer, M. S., and Rosen, S. D. (1990) Direct demonstration of the lectin activity of gp90MEL, a lymphocyte homing receptor, J Cell Biol 111, 1225-1232.
5. Nelson, R. M., Cecconi, O., Roberts, W. G., Aruffo, A., Linhardt, R. J., and Bevilacqua, M. P. (1993) Heparin oligosaccharides bind L- and P-selectin and inhibit acute inflammation, Blood 82, 3253-3258.
6. Koenig, A., Norgard-Sumnicht, K., Linhardt, R., and Varki, A. (1998) Differential interactions of heparin and heparan sulfate glycosaminoglycans with the selectins. Implications for the use of unfractionated and low molecular weight heparins as therapeutic agents, Journal of Clinical Investigation 101, 877-889.
7. Chang, J., Patton, J. T., Sarkar, A., Ernst, B., Magnani, J. L., and Frenette, P. S. (2010) GMI-1070, a novel pan-selectin antagonist, reverses acute vascular occlusions in sickle cell mice, Blood 116, 1779-1786.

1. A composition comprising an isolated pentosan polysulfate sodium (PPS) fraction wherein the composition has (a) improved or comparable P-selectin blocking activity, (b) improved bioavailability, and (c) no significantly greater anti-coagulant activity relative to unfractionated PPS.

2. The composition of claim 1, wherein the unfractionated PPS has an average molecular weight range of between about 1,221 Da-7,681 Da.

3. A composition comprising an isolated pentosan polysulfate sodium (PPS) fraction having the same properties as a composition produced by:
a. dissolving PPS in an aqueous solution;
b. adding an organic solvent in a stepwise manner to the dissolved PPS; and
c. isolating a precipitated PPS fraction.

4. The composition of claim 3, wherein the organic solvent is added in a stepwise manner to the dissolved PPS until the total concentration of the organic solvent is at least about 38% by volume.

5. The composition of claim 4, wherein steps (b) and (c) of claim 3 are repeated using progressively increasing concentrations of organic solvent comprising at least about 43%, 46%, 48%, and/or 50% by volume.

6. The composition of claim 3, wherein the stepwise manner comprises removing the supernatant and re-dissolving the precipitated PPS fraction.

7. The composition of claim 3, wherein the properties are (a) improved p-selectin blocking activity, (b) improved bioavailability, and (c) no significantly greater anti-coagulant activity relative to unfractionated PPS.

8. The composition of claim 7, wherein the unfractionated PPS has an average molecular weight range of between about 1,221 Da-7,681 Da.

9. The composition of claim 3, wherein the composition is produced by:
a. dissolving PPS in an aqueous solution;
b. adding an organic solvent in a stepwise manner to the dissolved PPS; and
c. isolating a precipitated PPS fraction.

10. The composition of claim 9, wherein the organic solvent is added in a stepwise manner to the dissolved PPS until the total concentration of the organic solvent is at least about 38% by volume.

11. The composition of claim 10, wherein steps (b) and (c) of claim 9 are repeated using progressively increasing concentrations of organic solvent comprising at least about 43%, 46%, 48%, and/or 50% by volume.

12. The composition of claim 9, wherein the stepwise manner comprises removing the supernatant and re-dissolving the precipitated PPS fraction.

13. The composition of claim 3, wherein the organic solvent is selected from the group consisting of methanol, ethanol, propanol, and butanol.

14. The composition of claim 3, wherein the organic solvent is methanol.

15. The composition of claim 1, wherein the isolated PPS fraction has a weight average molecular weight (Mw) of between about 3761 Da-4832 Da.

16. The composition of claim 15, wherein the isolated PPS fraction has a weight average molecular weight (Mw) of about 4274 Da.

17. The composition of claim 1, wherein the isolated PPS fraction has a polydispersity index of between about 1.237 Mw/Mn−1.142 Mw/Mn.

18. The composition of claim 17, wherein the isolated PPS fraction has a polydispersity index of about 1.167 Mw/Mn.

19. The composition of claim 1, wherein the composition exhibits reduced E-selectin and L-selectin blocking activity compared to P-selectin blocking activity.

20. The composition of claim 19, wherein (i) the composition exhibits less than 5% E-selectin blocking activity; or (ii) the composition exhibits less than 2% L-selectin blocking activity.

21. (canceled)

22. A pharmaceutical composition comprising the composition of claim 1.

23. The pharmaceutical composition of claim 22, further comprising one or more of an anionic surfactant, a cationic surfactant, an organic acid, and/or an antioxidant.

24. A method for treating sickle cell disease (SCD) in a subject in need thereof, comprising administering a clinically effective amount of the pharmaceutical composition of claim 22 to the subject.

25. The method of claim 24, wherein the pharmaceutical composition is administered orally.

26. (canceled)

27. A kit comprising:
a. the composition of claim 1; and
b. one or more pharmaceutically acceptable excipients, carriers, adjuvants or vehicles.

28. A method for detecting pentosan polysulfate sodium (PPS) or a PPS fraction in a biological sample, the method comprising:
a. contacting the sample with a protease;
b. extracting and precipitating the PPS or the PPS fraction in the sample;
c. contacting the sample with an antibody which binds to PPS or the PPS fraction, wherein the antibody is directly or indirectly capable of detection; and
d. detecting the antibody, thereby detecting the presence of PPS or the PPS fraction in the biological sample.

29. The method of claim 28, wherein the biological sample is blood or a product derived from blood.

30. The method of claim 29, wherein the biological sample is serum or plasma.

31. The method of claim 28, wherein
(i) the method has a Lower Limit of Detection (LLOD) (2× signal/background) of between about 0.5 ng/mL to about 10 ng/mL;
(ii) the antibody which binds to PPS or the PPS fraction is used in an ELISA assay;
(iii) the PPS is extracted and precipitated with chloroform and ammonium acetate; and/or
(iv) step (b) is performed before step (a).

32-35. (canceled)
